(19) United States
(12) Patent Application Publication
(10) Pub. No.: US 2013/0315928 A1
(43) Pub. Date: Nov. 28, 2013

(54) LUTEINIZING-HORMONE LIGAND AND LIGAND-GONADOTROPHIN COMPLEX

(71) Applicants: Marie-Christine Maurel, Tours (FR); Eric Reiter, Nouzilly (FR); Florian Guillou, Tours (FR); Nicolas Aubrey, Truyes (FR)

(72) Inventors: Marie-Christine Maurel, Tours (FR); Eric Reiter, Nouzilly (FR); Florian Guillou, Tours (FR); Nicolas Aubrey, Truyes (FR)

(21) Appl. No.: 13/988,203

(22) PCT Filed: Nov. 21, 2011
(86) PCT No.: PCT/IB2011/055210

§ 371 Date: Aug. 1, 2013

(30) Foreign Application Priority Data
Nov. 19, 2010 (FR) 1004503

(51) Int. Cl. C07K 16/26; A61K 47/48

(52) U.S. Cl. CPC C07K 16/26; A61K 47/48553
USPC 424/158.1; 530/389.2; 530/388.24; 530/391.1; 424/178.1; 530/387.3; 435/375

(57) ABSTRACT

The invention relates to a ligand of a luteinizing hormone (LH), characterized in that it comprises the paratope of an ovine anti-LH antibody of which the variable domain of the heavy chain contains the following CDRs: —VH-CDR1, defined by the sequence GYTFTNYW (SEQ ID NO: 13); —VH-CDR2, defined by the sequence IYPGGGYT (SEQ ID NO: 14); —VH-CDR3, defined by the sequence ARTPLYGSSYGGFAY (SEQ ID NO: 15); and the variable domain of the light chain contains the following CDRs: —VL-CDR1, defined by the sequence QGISNY (SEQ ID NO: 16); —VL-CDR2, defined by the sequence YTS; —VL-CDR3, defined by the sequence QQYSKLPWT (SEQ ID NO: 17). The invention also relates to a ligand-gonadotrophin (LH, hCG, FSH) complex. The ligand or the complex according to the invention can be used to induce ovulation in a female mammal.

The present invention relates to antibodies directed against luteinizing hormone (LH), and uses thereof.

Luteinizing hormone is a glycoprotein hormone in the gonadotropin family. This family comprises various hormones involved in the functioning of the genital glands, and gametogenesis; a distinction is made between the hypophyseal gonadotropins, which are produced in all mammalian species, and which comprise, besides LH, follicle-stimulating hormone (FSH), and the chorionic gonadotropins, which are produced by the placenta and only exist in certain mammalian species: human (hCG) and horse (eCG).

The gonadotropins have a common structure: they are formed from two glycoprotein chains (alpha and beta), bound noncovalently. Within one and the same species, the alpha chain is common to LH, to FSH, to TSH and possibly to the chorionic gonadotropin, and it is the beta chain that is responsible for the specificity of the hormone.

In females, FSH is responsible for the growth and differentiation of the oocytes and LH induces terminal growth of the oocytes and ovulation. In males, LH stimulates testosterone production.

In females, both LH and FSH are at very low plasma concentrations in the sexual rest period and outside of the ovulatory period. A peak in secretion of these hormones occurs in the preovulatory period, which leads to the initiation of ovulation. The gonadotropins are used in human and veterinary medicine for imitating the endocrine mechanisms governing the sexual cycles. For example, in sheep and goat breeding, eCG (which, in species other than the equine species, has a dual, LH and FSH activity) combined with a progestogen is used for inducing and synchronizing estrus and ovulation, as well as in the context of superovulation treatments.

Although these techniques are of proven efficacy, they pose an appreciable health risk, as the eCG is extracted from the plasma of pregnant mares. Moreover, in certain animals the repeated use of eCG induces an immune response that is reflected in secretion of anti-eCG antibodies in the plasma. In most cases this immune response leads to neutralization of the activity of eCG, and is reflected in reduced efficacy of the treatment. In a small number of animals, however, it was found that the anti-eCG antibodies produced had, in contrast, the property of potentiating the bioactivity of eCG. Polyclonal antibodies possessing this property are described in application EP1518863. However, these antibodies require the simultaneous administration of eCG.

The inventors have now obtained monoclonal antibodies produced against ovine endogenous LH, and they observed that certain of these antibodies were capable of potentiating its action.

These potentiating monoclonal antibodies are called respectively hereinafter 9A4 A7 D3 (also designated D3 hereinafter), 1A6 C4 G11 (also designated G11 hereinafter) and 9A4 D4 B6 (also designated B6 hereinafter).

The hybridoma producing the antibody 9A4 A7 D3 was deposited, in accordance with the Budapest Treaty, on 30 Jun. 2010 at the CNCM (Collection Nationale de Cultures de Microorganismes Institut Pasteur [National Collection of Cultures of Microorganisms, Pasteur Institute], 25 rue du Docteur Roux, 75724 Paris Cedex 15, France), under number CNCM I-4333.

The hybridoma producing the antibody 1A6 C4 G11 was deposited, in accordance with the Budapest Treaty, on 30 Jun. 2010 at the CNCM, under number CNCM I-4332.

The hybridoma producing the antibody 9A4 D4 B6 was deposited, in accordance with the Budapest Treaty, on 30 Jun. 2010 at the CNCM, under number CNCM I-4334.

Moreover, the inventors discovered that, surprisingly, two of these antibodies, namely 9A4 A7 D3 and 9A4 D4 B6, also possess an FSH potentiating effect.

The sequences of the variable regions of the heavy chain and of the light chain of these antibodies were determined. These sequences, as well as the polypeptide sequences deduced, are shown in Table I below (for the light chain and the heavy chain of 9A4 A7 D3), in Table II below (for the light chain and the heavy chain of 1A6 C4 G11) and in Table III below (for the light chain and the heavy chain of 9A4 D4 B6). The nucleotide sequences are also shown in the appended sequence listing under the numbers SEQ ID NO: 1, 3, 5, 7, 9 and 11 respectively, and the polypeptide sequences are also shown under the numbers SEQ ID NO: 2, 4, 6, 8, 10 and 12 respectively.

TABLE I
Antibody 9A4 A7 D3
Heavy chain (VH)

| | |
|---|---|
| Nucleotide sequence | GAGGTCCAACTGCAGGAGTCAGGAGCTGAGCTGGTAAGGCCTGGGACTTCAGTG |
| SEQ ID NO: 1 | AAGATATCCTGCAAGGCTTCTGGCTACACCTTCACTAACTACTGGCTAGGTTGG |
| | GTAAAGCAGAGGCCTGGACATGGACTTGAGTGGATTGGAGATATTTACCCTGGA |
| | GGTGGTTATACTAACTACAATGAGAAGTTCAAGGGCAAGGCCACACTGACTGCA |
| | GACACATCCTCCAGCACTGCCTACATGCAGCTCAGTAGCCTGACATCTGAGGAC |
| | TCTGCTGTCTATTTCTGTGCAAGAACCCCTCTCTACGGTAGTAGCTACGGGGGG |
| | TTTGCTTACTGGGGCCAAGGGACTCTGGTCACTGTCTCTGCAGA |
| Peptide sequence | EVQLQESGAELVRPGTSVKISCKASGYTFTNYWLGWVKQRPGHGLEWIGDIYPG |
| SEQ ID NO: 2 | GGYTNYNEKFKGKATLTADTSSSTAYMQLSSLTSEDSAVYFCARTPLYGSSYGG |
| | FAYWGQGTLVTVSA |

Light chain (VL)

| | |
|---|---|
| Nucleotide sequence | AAGACACAGACTACATCCTCCCTGTCTGCCTCTCTGGGAGACAGAGTCACCATC |
| SEQ ID NO: 3 | AGTTGCAGTGCAAGTCAGGGCATTAGCAATTATTTAAACTGGTATCAGCAGAAA |
| | CCAGATGGAACTGTTAAACTCCTGATCTATTACACATCAAGTTTACACTCAGGA |
| | GTCCCATCAAGGTTCAGTGGCAGTGGGTCTGGGACAGATTATTCTCTCACCATC |
| | AGCAACCTGGAACCTGAAGATATTGCCACTTACTATTGTCAGCAGTATAGTAAG |
| | CTTCCGTGGACGTTCGGTGGAGGCACCAAGCTGGAAATCAAAC |
| Peptide sequence | KTQTTSSLSASLGDRVTISCSASQGISNYLNWYQQKPDGTVKLLIYYTSSLHSG |
| SEQ ID NO: 4 | VPSRFSGSGSGTDYSLTISNLEPEDIATYYCQQYSKLPWTFGGGTKLEIK |

TABLE II
Antibody 1A6 C4 G11
Heavy chain (VH)

| | |
|---|---|
| Nucleotide sequence | GAGGTGAAGCTGCAGCAGTCAGGAGCTGAGCTGGTAAGGCCTGGGACTTCAGT |
| SEQ ID NO: 5 | GAAGATATCCTGCAAGGCTTCTGGCTACACCTTCACTAACTACTGGCTAGGTT |
| | GGGTAAAGCAGAGGCCTGGACATGGACTTGAGTGGATTGGAGATATTTACCCT |
| | GGAGGTGGTTATACTAACTACAATGAGAAGTTCAAGGGCAAGGCCACACTGAC |
| | TGCAGACACATCCTCCAGCACTGCCTACATGCAGCTCAGTAGCCTGACATCTG |
| | AGGACTCTGCTGTCTATTTCTGTGCAAGAACCCCTCTCTACGGTAGTAGCTAC |
| | GGGGGGTTTGCTTACTGGGGCCAAGGGACTCTGGTCACTGTCTCTGCAGA |
| Peptide sequence | EVKLQQSGAELVRPGTSVKISCKASGYTFTNYWLGWVKQRPGHGLEWIGDIYP |
| SEQ ID NO: 6 | GGGYTNYNEKFKGKATLTADTSSSTAYMQLSSLTSEDSAVYFCARTPLYGSSY |
| | GGFAYWGQGTLVTVSA |

Light chain (VL)

| | |
|---|---|
| Nucleotide sequence | GATATCCAGATGACACAGACTACATCCTCCCTGTCTGCCTCTCTGGGAGACAG |
| SEQ ID NO: 7 | AGTCACCATCAGTTGCAGTGCAAGTCAGGGCATTAGCAATTATTTAAACTGGT |
| | ATCAGCAGAAACCAGATGGAACTGTTAAACTCCTGATCTATTACACATCAAGT |
| | TTACACTCAGGAGTCCCATCAAGGTTCAGTGGCAGTGGGTCTGGGACAGATTA |
| | TTCTCTCACCATCAGCAACCTGGAACCTGAAGATATTGCCACTTACTATTGTC |
| | AGCAGTATAGTAAGCTTCCGTGGACGTTCGGTGGAGGCACCAAGCTGGAAATC |
| | AAAC |
| Peptide sequence | DIQMTQTTSSLSASLGDRVTISCSASQGISNYLNWYQQKPDGTVKLLIYYTSS |
| SEQ ID NO: 8 | LHSGVPSRFSGSGSGTDYSLTISNLEPEDIATYYCQQYSKLPWTFGGGTKLEI |
| | K |

TABLE III
Antibody 9A4 D4 B6
Heavy chain (VH)

| | |
|---|---|
| Nucleotide sequence | GAGGTGCAACTGCAGCAGTCTGGAGCTGAGCTGGTAAGGCCTGGGACTTCAGT |
| SEQ ID NO: 9 | GAAGATATCCTGCAAGGCTTCTGGCTACACCTTCACTAACTACTGGCTAGGTT |
| | GGGTAAAGCAGAGGCCTGGACATGGACTTGAGTGGATTGGAGATATTTACCCT |
| | GGAGGTGGTTATACTAACTACAATGAGAAGTTCAAGGGCAAGGCCACACTGAC |
| | TGCAGACACATCCTCCAGCACTGCCTACATGCAGCTCAGTAGCCTGACATCTG |
| | AGGACTCTGCTGTCTATTTCTGTGCAAGAACCCCTCTCTACGGTAGTAGCTAC |
| | GGGGGGTTTGCTTACTGGGGCCAAGGGACTCTGGTCACTGTCTCTGCA |
| Peptide sequence | EVQLQQSGAELVRPGTSVKISCKASGYTFTNYWLGWVKQRPGHGLEWIGDIYP |
| SEQ ID NO: 10 | GGGYTNYNEKFKGKATLTADTSSSTAYMQLSSLTSEDSAVYFCARTPLYGSSY |
| | GGFAYWGQGTLVTVSA |

Light chain (VL)

| | |
|---|---|
| Nucleotide sequence | GATATTGTGATGACGCAGGCTACATCCTCCCTGTCTGCCTCTCTGGGAGACAG |
| SEQ ID NO: 11 | AGTCACCATCAGTTGCAGTGCAAGTCAGGGCATTAGCAATTATTTAAACTGGT |
| | ATCAGCAGAAACCAGATGGAACTGTTAAACTCCTGATCTATTACACATCAAGT |
| | TTACACTCAGGAGTCCCATCAAGGTTCAGTGGCAGTGGGTCTGGGACAGATTA |
| | TTCTCTCACCATCAGCAACCTGGAACCTGAAGATATTGCCACTTACTATTGTC |
| | AGCAGTATAGTAAGCTTCCGTGGACGTTCGGTGGAGGCACCAAGCTGGAAATC |
| | AAA |
| Peptide sequence | DIVMTQATSSLSASLGDRVTISCSASQGISNYLNWYQQKPDGTVKLLIYYTSS |
| SEQ ID NO: 12 | LHSGVPSRFSGSGSGTDYSLTISNLEPEDIATYYCQQYSKLPWTFGGGTKLEI |
| | K |

The sequences coding for the CDRs of 9A4 A7 D3, of 1A6 C4 G11 and of 9A4 D4 B6 were also determined, from the above sequences of the heavy chains and of the light chains, using the IMGT/V-QUEST software (Giudicelli et al., Nucleic Acids Research 32, W435-W440, 2004; Brochet, X. et al., Nucl. Acids Res. 36, W503-508, 2008). These sequences are identical for the three antibodies.

The polypeptide sequences deduced are shown below in Table IV for the three antibodies 9A4 A7 D3, 1A6 C4 G11 and 9A4 D4 B6. They are also given in the appended sequence listing under the numbers SEQ ID NO: 13 to 17.

TABLE IV

| | |
|---|---|
| | Polypeptide |
| References | sequence |

Heavy chain

| | |
|---|---|
| VH-CDR1 (SEQ ID NO: 13) | GYTFTNYW |
| VH-CDR2 (SEQ ID NO: 14) | IYPGGGYT |
| VH-CDR3 (SEQ ID NO: 15) | ARTPLYGSSYGGFAY |

Light chain

| | |
|---|---|
| VL-CDR1 (SEQ ID NO: 16) | QGISNY |
| VL-CDR2 | YTS |
| VL-CDR3 (SEQ ID NO: 17) | QQYSKLPWT |

The present invention relates to a ligand of a luteinizing hormone (LH), characterized in that it comprises the paratope of an anti-ovine LH antibody whose heavy chain variable domain contains the following CDRs:

(SEQ ID NO: 13)
VH-CDR1, defined by the sequence GYTFTNYW;
(SEQ ID NO: 14)
VH-CDR2, defined by the sequence IYPGGGYT;
(SEQ ID NO: 15)
VH-CDR3, defined by the sequence
ARTPLYGSSYGGFAY;
and

the light chain variable domain contains the following CDRs:

(SEQ ID NO: 16)
VL-CDR1, defined by the sequence QGISNY;
VL-CDR2, defined by the sequence YTS;
(SEQ ID NO: 17)
VL-CDR3, defined by the sequence QQYSKLPWT.

The CDRs (complementarity determining regions) are the portions of the variable regions of an antibody involved in the specificity of antigen recognition.

“Anti-ovine LH antibody” as used here is defined as any antibody obtained by immunization of an animal with ovine LH, and capable of binding with the latter. This definition is not limited to antibodies capable of binding selectively with ovine LH, but also includes antibodies capable of also binding with LHs of other mammals, for example bovine, caprine, porcine or human LH; moreover, it also includes antibodies capable of also binding with one or more other gonadotropin(s) (ovine or of some other origin), such as notably FSH, or hCG. Moreover, the term “LH ligand” also includes ligands capable of also binding with one or more other gonadotropin(s).

Here, “gonadotropin” means any protein with gonadotropic activity, i.e. capable of stimulating the FSH and LH receptors, whether it is a natural protein or a recombinant protein. More particularly, the terms “LH” and “FSH” include, besides the natural LH or FSH, recombinant LH or FSH optionally modified to optimize their pharmacological properties. As a nonlimiting example, we may mention corifollitropin alfa, which is a chimeric FSH resulting from fusion of the carboxy-terminal peptide of the beta subunit of human chorionic gonadotropin (hCG) with the beta chain of human FSH, which has the effect of prolonging its half-life, and in consequence its duration of action, without affecting its FSH activity.

The inventors tested the binding capacities of the three anti-ovine LH antibodies 9A4 A7 D3, 9A4 D4 B6, and 1A6 C4 G11 with ovine, bovine, or porcine LH, ovine, bovine, porcine, or human FSH, as well as hCG, and found that they are capable of binding to all these gonadotropins.

The sequences of CDR1, CDR2 and CDR3 of the light chain, as well as the sequences of CDR1, CDR2 and CDR3 of the heavy chain are identical for the three monoclonal antibodies B6, D3 and G11. Moreover, the sequences of the frameworks FR2, FR3 and FR4 of the light chain, as well as the sequences of FR2, FR3 and FR4 of the heavy chain are identical.

However, the N-terminal sequences of FR1 of the VH and VL chains vary depending on the antibody in question:

Thus, in the case of antibody G11 (which binds to LH and to FSH, but only potentiates LH) the N-terminal sequence determined for region FR1 of the light chain is DIQMTQTTSS (SEQ ID NO: 18), and that determined for region FR1 of the heavy chain is EVKLQQSGAE (SEQ ID NO: 19).

In the case of antibody B6 (which binds to LH and to FSH, and potentiates these two gonadotropins), the N-terminal sequence determined for region FR1 of the light chain is DIVMTQATSS (SEQ ID NO: 20), and that determined for region FR1 of the heavy chain is EVQLQQSGAE (SEQ ID NO: 21).

In the case of antibody D3 (which binds to LH and to FSH, and potentiates these two gonadotropins), the N-terminal sequence determined for region FR1 of the light chain is KTQTTSS (SEQ ID NO: 22), and that determined for region FR1 of the heavy chain is EVQLQESGAE (SEQ ID NO: 23).

In the case of scFv B6, which is derived from antibody B6 and which has the same properties of binding to LH and to FSH and of potentiating these two gonadotropins as antibodies B6 and D3, the N-terminal sequence of region FR1 of the VH domain differs from SEQ ID NO: 21 by the presence of an N-terminal glutamine, instead of a glutamic acid.

Without this hypothesis being limiting, the capacity for potentiating FSH seems to be determined by the N-terminal sequence of region FR1 of the heavy chain, and notably by the nature of the amino acid in position 3. The presence of a lysine at this position seems to be associated with the capacity of the antibody for potentiating only the activity of LH, whereas the presence of a glutamine seems to be associated with the capacity of the antibody for potentiating both the activity of LH and that of FSH.

According to a first embodiment of an LH ligand according to the invention, said ligand potentiates LH but not FSH, and the N-terminal portion of the framework region FR1 of its heavy chain is defined by the sequence EVKLQQSGAE (SEQ ID NO: 19).

According to a second embodiment of an LH ligand according to the invention, said ligand potentiates LH and FSH, and the N-terminal portion of the framework region FR1 of its heavy chain is defined by the sequence X₁VQLQX₁SGAE (SEQ ID NO: 24), in which X₁ represents a glutamine or a glutamic acid, preferably a glutamine.

According to a preferred configuration of one or other of these embodiments, the N-terminal portion of region FR1 of the light chain of said ligand contains the sequence X₂TQX₃TSS (SEQ ID NO: 25), in which X₂ represents a methionine or a lysine and X₃ represents a threonine or an alanine; advantageously, said N-terminal portion is defined by the sequence DIX₄X₂TQX₃TSS (SEQ ID NO: 26), in which X₂ and X₃ are as defined above, and X₄ represents a glutamine or a valine.

As examples, said N-terminal portion can be defined by one of the following sequences:

| | |
|---|---|
| the sequence DIQMTQTTSS; | (SEQ ID NO: 18) |
| the sequence DIVMTQATSS; | (SEQ ID NO: 20) |
| the sequence DIQMTQATSS; | (SEQ ID NO: 27) |
| the sequence KTQTTSS. | (SEQ ID NO: 22) |

An LH ligand according to the first embodiment is for example a ligand containing region FR1 of the light chain and region FR1 of the heavy chain of antibody G11, and advantageously, the whole of the variable domains VH and VL of said antibody.

LH ligands according to the second embodiment of the invention are for example:
a ligand containing region FR1 of the light chain and region FR1 of the heavy chain of antibody D3, and advantageously, the whole of the variable domains VH and VL of said antibody;
a ligand containing region FR1 of the light chain and region FR1 of the heavy chain of antibody B6, and advantageously, the whole of the variable domains VH and VL of said antibody;
a ligand containing region FR1 of the variable domain VH and region FR1 of the variable domain VL of scFv fragment B6, and advantageously the whole of the variable domains VH and VL of said fragment scFv.

LH ligands according to the invention notably include:

a) the monoclonal antibody 1A6 C4 G11 produced by the hybridoma CNCM I-4332;

b) the monoclonal antibody 9A4 A7 D3 produced by the hybridoma CNCM I-4333;

c) the monoclonal antibody 9A4 D4 B6 produced by the hybridoma CNCM I-4334;

d) a Fab, Fab′, Fab′2 fragment of an antibody a), b) or c) above;

e) a recombinant protein comprising the paratope of an antibody a), b) or c) above.

The recombinant proteins according to the invention can notably be recombinant antibodies derived from the monoclonal antibodies a), b) or c) above, modified in order to reduce their immunogenicity in the animal or human to which they are intended to be administered.

A recombinant antibody according to the invention can for example be a chimeric antibody, i.e. a recombinant antibody conserving the variable domains of the monoclonal antibody from which it was derived, but whose constant domains have been substituted with those of another antibody, generally those of an antibody originating from the species to which the subject belongs to which the chimeric antibody must be administered.

It can also be a recombinant antibody in which the paratope of the original murine monoclonal antibody (called “donor” antibody), is transferred into an antibody (called “acceptor” antibody) originating from the species to which the subject belongs to which the recombinant antibody must be administered, replacing the paratope of said acceptor antibody. A recombinant antibody of this kind is called “humanized antibody” if the acceptor antibody is a human antibody. If the acceptor antibody is, for example, of ovine, caprine, bovine, porcine, etc. origin, the corresponding recombinant antibodies are called “ovinized antibody”, “caprinized antibody”, “bovinized antibody”, “porcinized antibody”, etc., respectively.

Various methods for carrying out this replacement are known per se. Those most commonly used are based on grafting CDRs, which consists of replacing the CDRs of the acceptor antibody with those of the donor antibody. In certain cases, grafting of CDRs can be completed by optimization of the regions FR, which consists of inserting amino acids of the regions FR of the donor antibody involved in its properties of binding to the antigen in place of the corresponding amino acids of the regions FR of the acceptor antibody, in order to optimize the antigen binding properties of the final recombinant antibody (cf. for example ROUTLEDGE et al., “Reshaping antibodies for therapy”, in Protein Engineering of Antibody Molecules for Prophylactic and Therapeutic Applications in Man, 13-44, Academic Titles, Nottingham, England, 1993, or ROGUSKA et al., Protein Engineering, 9(10): 895-904, 1996).

Recombinant antibodies according to the invention are preferably immunoglobulins of class IgM, and notably of Kappa isotype.

Recombinant proteins according to the invention can also be antibody fragments comprising the paratope of an antibody a), b), or c) above. It can notably be fragments Fab, Fab′, Fab′2, Fv, dsFv, or scFv, or else diabodies, triabodies or tetrabodies.

The monovalent Fab fragments each contain a light chain and the first half of a heavy chain joined together by a disulfide bridge. The divalent Fab′2 fragments comprise two Fab fragments and a part of the hinge region. The monovalent Fab′ fragments result from cleavage of the disulfide bridge in the hinge region of the Fab′2 fragments.

The Fv fragments consist of the variable domains of the VH and VL chains of an antibody, attached to one another by hydrophobic interactions. The dsFv fragment consists of a VH::VL dimer attached by a disulfide bridge. The scFv fragments consist of the variable portions of the heavy and light chains of an antibody, joined together via a flexible peptide linker (Clackson et al., Nature, 352: 624-628, 1991), thus forming a single-chain protein. The fragments Fv, dsFv, and scFv are monovalent. The diabodies, triabodies and tetrabodies are bi-, tri-, or tetravalent forms, resulting from the multimerization of two, three, or four scFv fragments, respectively.

If necessary, these antibody fragments can be combined with molecules for prolonging their plasma half-life on administration in vivo; they can for example be fused with a water-soluble polypeptide of sufficient molecular weight so that the molecular weight of the fusion polypeptide thus obtained is above the renal filtration threshold, or else conjugated with a polyol, for example polyethylene glycol.

According to a preferred embodiment of a ligand of the ovine LH according to the invention, it is an scFv fragment.

As a nonlimiting example, the peptide sequence of an scFv fragment according to the invention, derived from the antibody CNCM I-4334, is shown in the appended sequence listing under the number SEQ ID NO: 28.

Fab and Fab′2 fragments can be obtained from an antibody according to the invention, by enzymatic digestion, by papain in the case of a Fab fragment, and by pepsin in the case of a Fab′2 fragment. The Fab′ fragments can be obtained from Fab′2 fragments by cleavage of the disulfide bridge in the hinge region.

These fragments can also be obtained, as well as the recombinant antibodies, the fragments Fv, dsFv, scFv and their multivalent derivatives, by the classical techniques of genetic engineering, such as those described by SAMBROOK et al. (MOLECULAR CLONING, A LABORATORY MANUAL, 2nd Ed., Cold Spring Harbor Laboratory Press, Cold Spring Harbor, N.Y., 1989).

Polynucleotides coding for the variable regions of the monoclonal antibodies 9A4 A7 D3, 1A6 C4 G11 and 9A4 D4 B6 can be obtained by cloning said regions from cDNA databases of the hybridomas CNCM I-4333, CNCM I-4332 and CNCM I-4334. They can also be prepared fully or partially by synthesis of nucleic acids, starting from the nucleotide sequences of said variable regions.

The present invention also relates to any nucleic acid molecule coding for a ligand according to the invention, as well as any recombinant vector, notably any expression vector, comprising said nucleic acid molecule.

Nucleic acid molecules according to the invention can advantageously comprise, besides a sequence coding for a recombinant protein according to the invention, a sequence coding for a signal peptide permitting secretion of said protein; they can also comprise one or more sequence(s) coding for one or more marker peptide(s) permitting detection and/or facilitating purification of said protein.

Expression vectors according to the invention comprise at least one nucleic acid sequence coding for a protein according to the invention, associated with elements controlling transcription and translation that are active in the host cell selected. Host vectors usable for constructing expression vectors according to the invention are known per se, and will be selected notably as a function of the host cell that is to be used.

The present invention also relates to any cell expressing a ligand of the ovine LH according to the invention. This notably includes the hybridomas CNCM I-4333, CNCM I-4332 and CNCM I-4334, as well as the host cells transformed with a nucleic acid molecule according to the invention.

Host cells usable in the context of the present invention can be prokaryotic or eukaryotic cells. The construction of expression vectors according to the invention and the transformation of the host cells can be carried out by the classical techniques of molecular biology.

The invention also relates to a method of producing an LH ligand according to the invention, characterized in that it comprises culturing at least one cell according to the invention, and recovering said ligand from said culture.

If the ligand is secreted, it can be recovered directly from the culture medium; otherwise preliminary lysis of the cells will be employed, or recovery of the periplasm in the case of expression in Escherichia coli.

The ligand can then be purified from the ascitic fluid, from the culture medium or from the cellular lysate by conventional procedures, known per se by a person skilled in the art, for example by fractional precipitation, notably precipitation with ammonium sulfate, electrophoresis, gel filtration, affinity chromatography, ion exchange chromatography etc.

The ligands according to the invention are usable in all cases where it is desired to potentiate the LH activity, not only of ovine LH but more generally of any natural or recombinant LH recognized by said ligands. The ligands containing the paratope of the antibodies D3 and B6 make it possible, moreover, to potentiate the FSH activity of any natural or recombinant FSH recognized by said ligand.

The invention also relates to a complex formed by a ligand according to the invention and a gonadotropin capable of binding to said ligand, and notably a gonadotropin, natural or recombinant, whose action is potentiated by said ligand.

Complexes according to the invention notably include:
a complex of a ligand according to the invention with LH;
a complex of a ligand according to the invention with hCG;
a complex of a ligand according to the invention, derived from one of the antibodies D3 or B6, with FSH.

The complexes according to the invention can be obtained by simple incubation of a ligand according to the invention with the gonadotropin selected. Administration of a complex according to the invention makes it possible to amplify the activity of the complexed gonadotropin, and consequently to decrease both the dose of gonadotropin to be injected and the number of injections required to give the same physiological response, or even a better response, relative to the same gonadotropin used alone.

The ligands or the ligand-gonadotropin complexes according to the invention can be used in vitro for analyzing the potentiation of the bioactivity of said gonadotropins at the level of their target receptor(s). They can be used as research tools for studying the changes induced by the phenomenon of potentiation on activation of the signalling pathways of the target receptors, on internalization of these receptors and on their desensitization.

They can also be used in vivo notably as a medicinal product, for increasing the bioactivity of LH, or, in the case of the ligands derived from the antibodies B6 and D3, for increasing both the bioactivity of LH and that of FSH.

For this, they are used either for amplifying LH activity or the endogenous LH and FSH activity in the subject to be treated, or for amplifying the activity of an exogenous LH or FSH. In the first case, the ligand-endogenous gonadotropin complex forms after injection of the ligand in the subject to be treated, the antibody playing the role of nonhormonal substitute. In the second case, the ligand plays the role of potentiator of the gonadotropin injected; the gonadotropin and the ligand can either be mixed beforehand to form a ligand-gonadotropin complex prior to administration, or administered separately.

The ligands or the ligand-gonadotropin complexes according to the invention can be used in particular:
for inducing ovulation in a female mammal. Their administration can notably make it possible to mimic the peak of secretion of LH and, if desired, of FSH, which normally occurs in the preovulatory period, and thus initiate ovulation;
in the context of treating pathological states resulting from low circulating levels of LH and FSH, for example disorders resulting from hypophyseal insufficiency;
for treating subjects, male or female, with hyporeceptivity of the gonads to LH and FSH.

The ligands or the ligand-gonadotropin complexes according to the invention can be used in humans or in various mammals, notably farm animals (for example sheep, bovines, goats, pigs, equines), or pets (for example dogs or cats).

The ligands or the ligand-gonadotropin complexes according to the invention will preferably be administered by injection, which can equally be intramuscular, intravenous, intraperitoneal, intradermal, intraorbital or subcutaneous without altering the potentiating effect of the ligand.

The present invention will be better understood from the rest of the description given below, which refers to nonlimiting examples of preparation and use of LH ligands according to the invention.

### EXAMPLE 1

### Measurements In Vitro of the Potentiating Effect of the Mabs and Characterization of the Potentiating Mabs

The potentiating effects of the monoclonal antibodies (MAb) secreted by the hybridomas CNCM I-4333, I-4332 and I-4334 were measured by in vitro bioassay specific to LH activity, performed with the MLTC cell line, which stably expresses the LH receptor. The response measured is cAMP secretion after 3 hours of stimulation at 37° C. with LH alone or previously incubated with the supernatant of the hybridomas.

Moreover, the potentiating effect on FSH activity was measured by a bioassay in vitro, performed with the LTK cell line (mouse fibroblast line), which stably expresses the FSH receptor, or on bovine granulosa cells in suspension. The response measured is cAMP secretion after 3 hours of stimulation at 37° C. with FSH alone or previously incubated with the supernatant of the hybridomas.

By comparing the biological response obtained in the presence of the hormone alone and that obtained with the hormone previously incubated with a hybridoma supernatant, assayed beforehand for anti-LH antibody, it is possible to determine whether the latter exerts a potentiating effect, an inhibitory effect, or no effect.

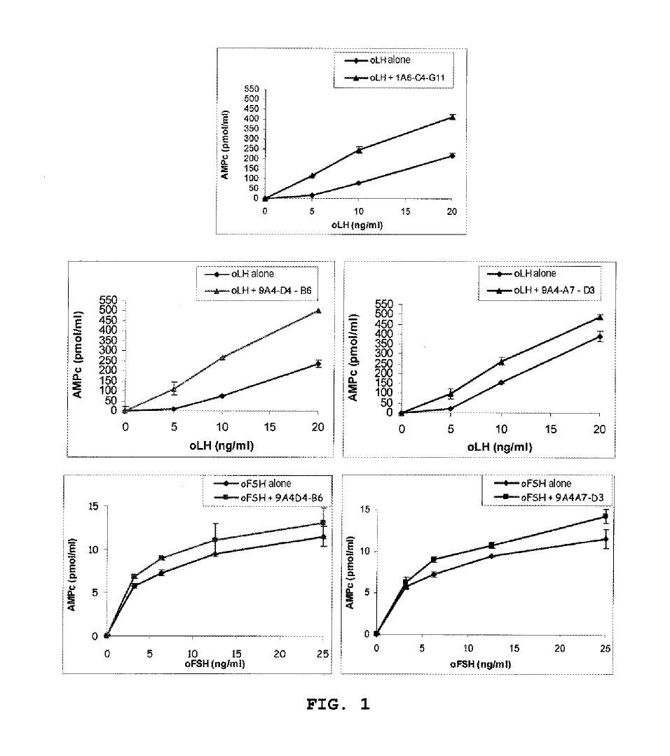

The results of the bioassay are presented in FIG. 1.

Among the 3 hybridomas, one (I-4332) is a secretor of antibodies potentiating the activity of oLH strictly (1A6-C4-G11), the potentiating effect ranging from 700 to 1300% for the point 5 ng/ml of hormone. Remarkably, the other two hybridomas are secretors of antibodies potentiating both the activity of oLH and of oFSH (9A4-A7-D3 and 9A4-D4-B6), this potentiating effect being 150% for the points 6.2 ng/ml, 12.5 ng/ml and 25 ng/ml of hormone. These 3 antibodies are all of isotype IgM.

The nucleotide sequences of the variable region of the heavy chains and of the light chains of antibodies 9A4 A7 D3, 1A6 C4 G11 and 9A4 D4 B6 were determined as follows. The total RNAs were extracted from 10⁹ hybridoma cells, using the reagent RNABLe® (EUROBIO, France), following the protocol recommended by the manufacturer. The RNAs were then isolated, and spectrophotometry at 260/280 nm was used for determining the RNA concentration of the samples. Using the RT-PCR (“reverse transcription polymerase chain reaction”) technique, the complementary sequence of DNA was obtained from each RNA strand. The reaction mixture was composed of 4 μl of RNA at 2 μg/ml with addition of 20 μl of Oligo dT at 100 ng/μl and 38.2 μl of MilliQ water. After heating the sample for 5 minutes at 70° C., 20 μl of buffer 5× and 10 μl of dNTP were added. This volume was supplemented with 2 μl of reverse transcriptase and 3.2 μl of RNAse and the tube was left at 42° C. for 1 h. The reaction mixture used was composed of 2 μl of MgCl₂ at 25 mM, 8 μl of the four dNTPs at 2.5 mM, about 1 U of Taq Polymerase® and 5 μl of reaction buffer 10×. The two “sense” and “antisense” primers (1.5 μl) were then added as well as the cDNA (3 μl) and then the final volume was made up to 50 μl with MilliQ water. Nine primer pairs were used for amplifying the VL and two primers were used for the VH. Their sequences are described by Peter et al. (The Journal of Biological Chemistry, 278, 36740-36747, 2003) and by Mousli M. et al. (FEBS Letters 442: 183-188, 1999).

The number of PCR cycles was 30, each comprising 1 minute at 90° C., 1 minute at 47° C. and then 3 minutes at 72° C.

The PCR products were analyzed by electrophoresis on 2% agarose gel stained with ethidium bromide, purified with the “QIAquick PCR Purification” kit (QIAGEN, the Netherlands) and sequenced. Multiple alignment of the VH and VL sequences obtained was performed using the Multalin software (F. Corpet, Nucl. Acids Res., 16 (22), 10881-10890, 1988).

The amino acid sequences of the hypervariable loops CDR1, CDR2 and CDR3 of the variable regions of antibodies 9A4 A7 D3, 1A6 C4 G11 and 9A4 D4 B6 were deduced from the oligonucleotide sequences of the heavy chains and of the light chains above, by means of the IMGT/V-QUEST base. Alignment and delimitation of the framework regions and CDRs of the variable regions were performed according to the referencing of the base FR-IMGT and CDR-IMGT (IMGT/3Dstructure-DB; Kaas Q et al., Nucleic Acid Research, 32: 208-210, 2004).

### EXAMPLE 2

### Measurement of the Potentiating Effect of the Mabs In Vivo, in the Rat

10 mg of each monoclonal antibody from Example 1 was produced by culture in vitro. After purification and concentration, they were tested in vivo.

The rat model was chosen because it is the international reference used by the pharmacopeia for measuring the activity of the gonadotropic hormones.

As the anti-oLH antibodies obtained do not cross with the rat LH, a human exogenous hormone, hCG, was used for testing the potentiating effect of these antibodies. The hormone hCG is recognized very well by the anti-oLH antibodies and offers the advantage of having strict LH activity. Moreover, it is readily available commercially in a form that is very pure, and inexpensive.

Two reference bioassays used by the pharmacopeia for determining LH activity were therefore used: one in the male (Scobey M J et al., 2005, Reprod. Biol. Endocr. 3: 61) and the other in the female (Parlow A F, 1958, Fed. Proc. 17: 402).

Assay in the female is the subject of Example 3.

In the male, the bioactivity of LH or of hCG is quantified relative to the increase in weight of the seminal vesicles. Young rats aged 25 days are injected with the hormone alone (1.5 IU) or previously incubated with the antibody, once daily for 4 days and then sacrificed on the 5th day to measure the weight of the seminal vesicles. This varies in proportion to the activity of the LH, development of the seminal vesicles being very androgen-dependent.

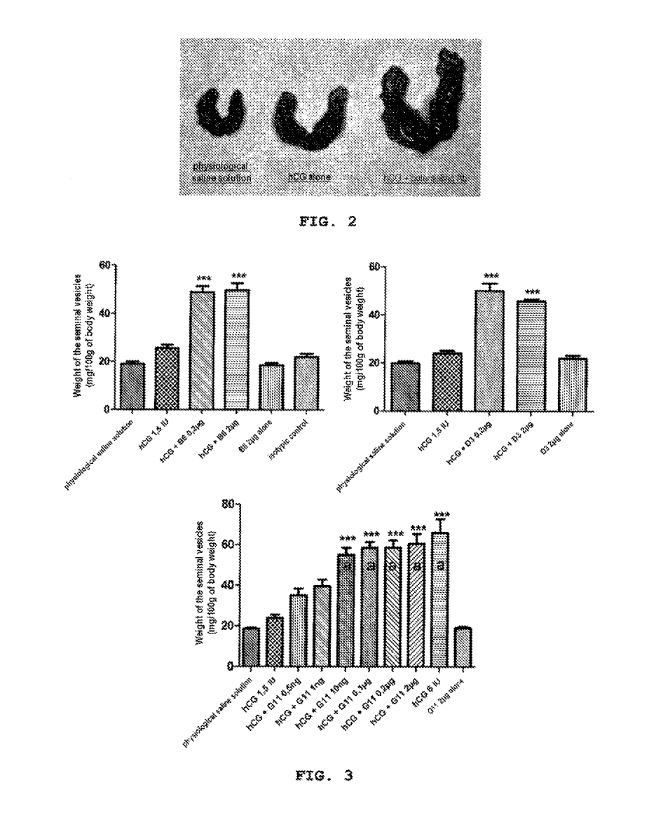

FIG. 2 shows the potentiating effect exerted by the potentiating antibody B6 on the bioactivity of hCG, observing the size of the seminal vesicles of a control rat treated with physiological saline solution, of a rat treated with 1.5 IU of hCG and of a rat treated with 1.5 IU of hCG preincubated with 2 μg of antibody B6.

FIG. 3 shows the potentiating effect of the MAbs B6, D3 and G11 on the increase in weight of the seminal vesicles. These results were obtained with batches of 8 rats and each experiment was repeated twice. In the rats treated with the hCG/MAb complex, the weight of the seminal vesicles doubled relative to the weight of the vesicles of rats treated with the hormone alone. Statistical analysis was performed with the GraphPad Prism software (GraphPad PRISM Software; GraphPad, San Diego, Calif.) by one-way analysis of variance and by the Bonferroni test (“Bonferroni's Multiple Comparison Test”). This showed that, for the three MAbs (B6, D3, G11), the weight of the seminal vesicles obtained in the batch treated with hCG 1.5 IU+MAb at 0.2 μg or 2 μg, by injection, is statistically very different from that of the batch treated with hCG alone (p<0.001). The rats treated with the antibody alone or with an isotypic control antibody (normal IgM) have a weight of the seminal vesicles equal to that of the control rat, indicating that the antibody alone does not have an effect.

The potentiating effect is obtained with very low doses of antibodies. An increase in weight of the seminal vesicles in the rats treated with the hCG+MAb G11 complex is observed starting from a dose of 0.5 ng of MAb. At doses of 0.5 and 1 ng of the MAb G11, the increase in weight shows a potentiating tendency but it is not significant. The potentiating effect of the complex becomes very significant (p<0.001) starting from the dose hCG 1.5 IU+MAb 10 ng by injection. It should be noted that the weights obtained with the doses hCG 1.5 IU+10 ng-0.1 μg-0.2 μg or 2 μg of the MAb G11 respectively are not statistically different from one another. Moreover, they are not different from the weight of the seminal vesicles obtained in rats treated with 6 IU of hCG alone. This means that the complex 10 ng of the MAb G11+hCG 1.5 IU is capable of inducing the same level of stimulation as 6 IU of hCG alone: its potentiating effect makes it possible to multiply the effect of the hormone alone by a factor of 4. This effect is almost maximal starting from 10 ng of MAb, reflecting very great sensitivity and efficacy of this MAb. Identical results were obtained with the MAbs B6 and D3 (not shown).

Absence of side effects following the treatments with the complexes was verified by weighing the testes and the epididymides of the animals and by histological examination of the gonads. The latter was perfectly normal and no abnormality was found in the weights of the testes and epididymides.

### EXAMPLE 3

### Measurement of the Potentiating Effects of the Mabs In Vivo, in the Female Rat

In the female rat, the bioactivity of LH is quantified from the drop in the level of ascorbic acid present in the ovaries, according to Parlow's determination (1958, cited above). After pretreatment with hCG on D0 and with eCG on D2 to luteinize the ovaries, the female rats are treated on D8, either with hCG alone or with hCG previously incubated with an antibody.

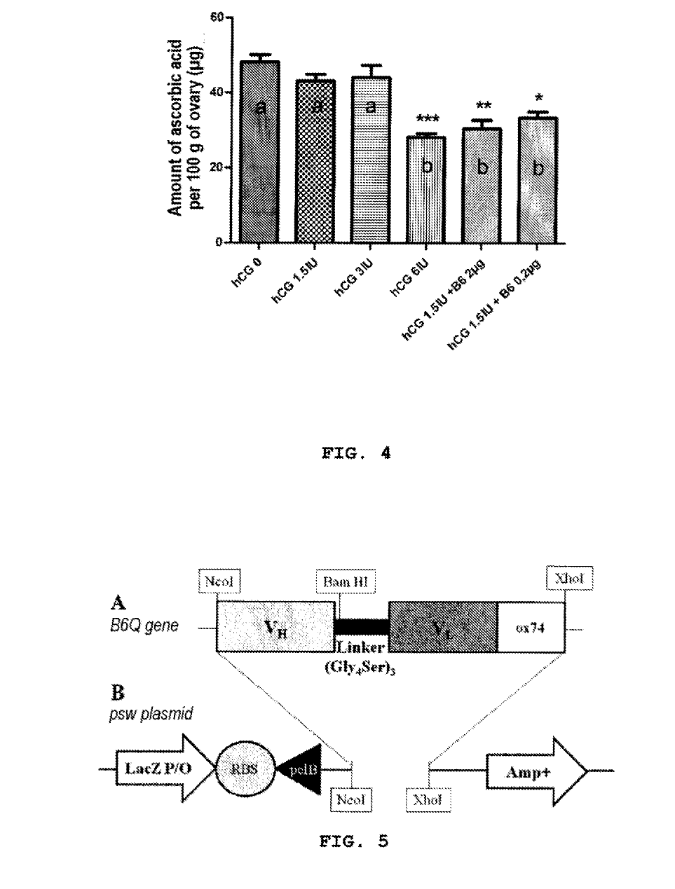

FIG. 4 shows the potentiating effect exerted by the MAb B6 in the female rat. Statistical analysis was performed with the GraphPad Prism software (GraphPad PRISM Software; GraphPad, San Diego, Calif.) by one-way analysis of variance and by the Bonferroni test (Bonferroni's Multiple Comparison Test). A significant drop in the level of ascorbic acid is observed in the ovaries of the rats treated with the complex hCG 1.5 IU+B6 2μg (p<0.01) or 0.2 μg (p<0.05) and hCG 6 IU alone (p<0.001). The results obtained show that the complex hCG 1.5 IU+antibody leads to a decrease in the level of ascorbic acid equivalent to a dose of 6 IU of hCG injected alone. The effect of the complex is therefore equivalent to that of a 4 times higher dose of hCG.

The same type of result was obtained with the other two MAbs (results not shown). No MAb exerts an effect when it is injected alone.

In conclusion, the results in examples 2 and 3 demonstrate that a potentiating effect of the hormone/antibody complex is very clearly exerted in vivo, leading to an amplified steroidogenic response in the target organs. They thus demonstrate unambiguously that the concept of potentiation of the activity of a gonadotropic hormone is applicable in vivo both in the male rat and in the female rat.

### EXAMPLE 4

### Construction of the scFv Fragments

The synthetic gene of the scFv B6 was prepared on the basis of the sequences of the antibody B6 with two modified amino acids: amino acid 1 (QVQ instead of EVQ) of framework 1 of the heavy chain and amino acid 3 (DIQ instead of DIV) of framework 1 of the light chain. This synthetic gene was inserted in a plasmid per4-TOPO and contains the restriction sites NcoI and XhoI at its 2 ends.

The synthetic gene is shown schematically in FIG. 5A. The codons were optimized for expression of the scFv in E. coli. The sequence of the gene was designed so as to be able to obtain the VH domain and the VL domain joined by a peptide linker. The latter consists of glycine and serine residues to endow the peptide linker with flexibility and resistance to proteases (Bird et al., Science, 242(4877): 423-426, 1988). When this linker is larger than 12 amino acids, the variable domains combine in their original conformation to form the antigen binding site (Whitlow et al., Protein Eng., 7(8): 1017-1026, 1994). At the 3′ end of the construct, there is a sequence coding for a 14-residue peptide. This flag peptide MRC-OX74 fused on the C-terminal of the recombinant protein facilitates its detection (Cyster et al., European Journal of Immunology, 22(10): 2565-2572, 1992).

The two restriction sites, NcoI and XhoI, at the ends of the synthetic gene permit its insertion in the plasmid pSW1 (FIG. 5B).

The plasmid pSW1 was used for constructing and expressing the gene coding for the recombinant antibody fragment. The plasmid contains, under the control of a LacZ inducible promoter, the signal sequence pelB, to which the sequence corresponding to the synthetic gene that has been developed is fused, in the reading frame. The signal sequence pelB will allow addressing of the scFv in the periplasm. The plasmid also contains a ribosome binding site (RBS) and an ampicillin resistance gene (Amp+).

The synthetic gene was excized from pCR4-TOPO using 0.5 μl of the endonucleases PstI and XhoI (Promega, USA) per 6 μl of plasmid for 1.5 h at 37° C. in the buffer recommended by the supplier. The same digestion was performed on the plasmid pSW1. The products obtained were then analyzed by agarose gel electrophoresis in the presence of ethidium bromide.

The bands of interest were isolated and purified from the agarose gel and the presence of the insert or of the digested vector and their concentration were verified with deposition of 2jtl of the eluate by electrophoresis on 1% agarose gel.

The vector pSW1-scFvB6 is obtained by ligation of the insert B6 with the plasmid pSW1. For this, in a reaction volume of 10 μl, the insert (2 μg) is incubated with 6 μg of plasmid cleaved and dephosphorylated in a ligation buffer, to which 1.5 μl of water and 0.5 μl of T4 DNA ligase (PROMEGA, USA) are added. In parallel, a control is performed without the insert. The whole is held at 15° C. for 14 h and then at 4° C. for 4 h.

The E. coli strain HB2151 is used for bacterial transformation. The latter is carried out, conventionally, from a ligation product or from plasmids already purified. The HB2151 strain of E. coli is made competent chemically using 1M calcium chloride. The plasmid or the ligation product (2 μl) is contacted with 200 μl of these bacteria for 30 minutes at 4° C. Next, the bacteria are submitted to thermal shock for 90 seconds in a bath at 42° C. The bacteria are immediately put back in the ice for 2 minutes for incorporation of the plasmid. Each transformation is diluted in 800 μl of LB (Luria Bertani) culture medium and incubated for 45 minutes at 37° C. with rotary stirring (200 rpm) to allow expression of the ampicillin resistance gene. After incorporating the plasmid, the bacteria reseal their wall and express the ampicillin resistance gene. They are then spread on a Petri dish containing the solid medium and are incubated overnight at 37° C. The colonies formed are then analyzed.

After transformation, the positive clones were selected on solid medium composed of agar in the presence of ampicillin. The bacteria selected are cultured for extracting the recombinant plasmids from them. To verify the presence of the insert, the plasmid DNA was digested with the same restriction enzymes as those used for cloning. The migration of the products of this digestion on 1% agarose gel revealed 2 clones (called 26 and 27) possessing the insert. The latter were sequenced in order to verify that the reading frame of the sequence is in phase and that the insert does not have any mutation.

### Bacterial Culture

The positive Escherichia coli bacteria were cultured in aerobiosis at 37° C., either in liquid medium with stirring (200 rpm), or in solid medium. The LB culture media were sterilized in an autoclave, and to obtain the solid media, 15 g of agar was added per liter of LB. The antibiotic for selecting the recombinant bacteria is ampicillin. It was added at a concentration of 100 μg per ml. The E. coli strains are stored at 4° C. in solid medium for a maximum time of 4 weeks. For longer-term storage, the bacterial strains can be stored at −80° C. in LB containing 15% (v/v) of sterile glycerol. The density of bacteria in liquid culture medium can be estimated by measuring the absorption at 600 nm: 1 unit of OD at 600 nm=8×10⁸ bacteria/ml.

### Bacterial Induction

Bacterial induction with LB culture medium was performed by adding IPTG (isopropyl β-D-1-thiogalactopyranoside), when the culture is in the stationary growth phase with an optical density above 1.2. A preculture was started with the bacterial colony of interest the previous day in LB culture medium containing ampicillin. The next day, a culture of larger volume (500 ml) was seeded by adding the preculture at 1:1000. Induction is effected with 0.84 mM of IPTG when the culture reaches an OD above 0.6. The culture was incubated with stirring (130 rpm) for 16 hours at 17° C.

After 16 hours of expression of the recombinant protein, the periplasmic proteins are isolated by controlled osmotic shock. Good production of scFv was verified on SDS-PAGE gel and then by Western blotting in denaturing conditions. This test is sufficiently sensitive to demonstrate good production and export of the recombinant protein in the periplasm of the bacteria.

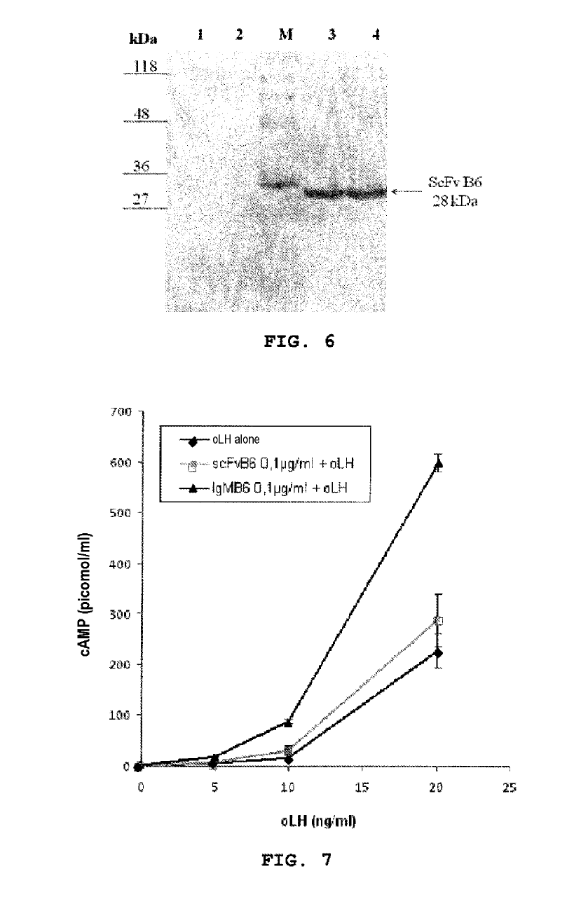

FIG. 6 shows the results of Western blot analysis of expression of the scFv B6 by E. coli. Tracks 1 and 2 correspond to an expression system that did not produce the scFv B6 (pHEN plasmid in the BL21 strain of E. coli). Tracks 3 and 4 correspond respectively to clones 26 and 27 that express the scFv B6. Track M: marker of molecular weight in kDa. The presence of a single band with a molecular weight of about 28 kDa is observed.

The protein from this band at 28 kDa is recognized by the anti-OX74 antibody in immunoblotting and thus shows that the protein produced corresponds to the scFv B6.

Extraction of the scFv

All the steps of extraction and dialysis were carried out at 4° C. To extract the recombinant proteins from the periplasm of the bacteria, the cultures are centrifuged at 5000 rpm for 20 minutes. To disrupt the external membrane, the bacterial pellet was taken up in a volume of TES (Tris-HCl 30 mM, EDTA 1 mM, sucrose 20%, pH 8.5) corresponding to 1:50 of the volume of the bacterial culture. The sample was left on ice for 15 mM and vortexed at regular intervals. This step was repeated a second time using the same lysis buffer diluted to ¼ and this time representing 1:33.33 of the volume of the culture. After centrifugation at 10000 rpm for 30 minutes, the periplasm was dialyzed overnight with stiffing against 5 liters of PBS (NaCl 0.14 M, KCl 13 mM, KH₂PO₄ 9 mM, Na₂HPO₄ 50 mM, pH 7.4).

Purification of the scFv B6

The Carboxyl-Adembeads kit (Ademtech, France) was used for purifying the scFv B6 from the preparation of periplasmic proteins following the protocol supplied by the supplier. The carboxyl group of the magnetic beads used in this kit was activated, thus making it possible to create a peptide bond with the anti-OX74 antibody. The periplasm was then incubated with this preparation for 3 h at 37° C., the scFv B6 was recognized by the fixed antibody owing to the presence of the OX74 flag peptide. The proteins retained are eluted with a solution of glycine-HCl 0.1 M at pH 2.0. 1-ml fractions were collected and the pH was adjusted immediately to a value of 7.5 by adding 50 μl of Tris 1 M. The eluted fractions whose absorbance at 280 nm is above 0.1 were combined and then dialyzed against PBS buffer overnight and were then concentrated on Minicon® concentrators (MILLIPORE, Ireland).

The peptide sequence of the scFv fragment B6 is shown in the appended sequence listing under the number SEQ ID NO: 28. This sequence SEQ ID NO: 28 does not include the flag peptide MRC-OX74.

The sequence of the gene coding for the scFv B6 can be modified by directed mutagenesis in order to obtain, in the peptide sequence, a proline residue (P) in place of a threonine residue (T), in position 145 of SEQ ID NO: 28. This mutation endows the scFv with the property that it can be recognized by protein L and can thus be purified by affinity chromatography on protein L.

### Protein Analysis

In this study, the SDS-PAGE technique was used for characterizing the presence of the scFv in the periplasm of the bacteria. The concentration of the acrylamide gel (12%) is selected in relation to the size of the protein of interest (Sambrook et al., 1989, cited above). Before being deposited, the sample was diluted in loading buffer (Tris-HCL 100 mM, pH 6.8, SDS 4%, β-mercaptoethanol 1%, bromophenol blue 0.2%, glycerol 20%) and denatured at 95° C. for 5 minutes. Migration of the proteins was carried out in an electrophoresis buffer at 60 V in the concentration gel and then at 150 V when the proteins penetrated into the separation gel. The gel was then incubated in a solution of Coomassie Blue (Coomassie Blue 0.25% (w/v), methanol 50% (v/v), acetic acid 10% (v/v)) for at least 30 minutes with stirring. The excess staining was removed by successive washings in a bleaching solution (ethanol 25% (v/v), acetic acid 7% (v/v)). The blue bands present on the gel correspond to the proteins contained in the sample. The proteins that were separated by electrophoresis were transferred passively overnight, on a nitrocellulose membrane in contact with the gel. The whole is surrounded by several thicknesses of Whatman paper soaked in the transfer buffer (Tris 25 mM pH 8.3, glycine 129 mM, SDS 0.01% (w/v), methanol 20% (v/v)).

At the end of transfer, the free sites of the nitrocellulose membrane were saturated for one hour at room temperature in a solution of transfer buffer with 5% (w/v) of dried milk. The membrane was then incubated for one hour with culture medium supernatant containing the anti-OX74 antibody specific to the peptide added at the C-terminus of the scFv. After washing three times in the transfer buffer, the membrane was incubated for one hour with a solution, which this time contained the secondary antibody (phosphatase-coupled mouse anti-IgG antibody). Finally the membrane was washed three times, and development was carried out by incubating for a few seconds with a substrate coupled to alkaline phosphatase (BCIP/NBT, (bromochloroindolyl phosphate/NitroBlue Tetrazolium)) diluted to half in the development buffer.

Analysis of the Specificity of the scFv with Respect to LH and Quantification by ELISA

The specificity of the scFv with respect to oLH was measured by ELISA. For this, the periplasms of clones 26/27 and a control periplasm without molecules recognizing oLH were tested.

For carrying out the ELISA assay, 100 μl of a solution of oLH at 1 μg/ml (diluted in 0.1 M carbonate-bicarbonate buffer, pH 9.6) is deposited per well of a plate (Nunc Immuno Plate) for 1 h at 37° C. and then overnight at 4° C. The residual sites are blocked with a saturated solution with 100 μl of a solution of PBS—Tween 0.1%—BSA 1%, at 37° C. for 45 minutes. The different dilutions of periplasms are deposited in duplicate at a rate of 100 μl per well, and the plate is incubated at 37° C. for 1 h. The primary antibody of the scFvB6 is deposited by adding 100 μl of a solution composed of culture supernatant, containing the anti-OX74 antibody diluted to a third in PBS buffer—Tween 0.01%—BSA 0.01% after washing 5 times with PBS—Tween 0.1%. This antibody is recognized by a peroxidase-coupled mouse IgG diluted to a 2500th in the same buffer as the primary antibody; 100 μl was added per well. After the plate has been washed another 5 times, it is held at 37° C. for 1 h. Then 100 μL of TMB (3,3′,5,5′-tetramethylbenzidine, Kirkegaard & Perry Laboratories Inc., USA), a chromogenic substrate of peroxidase, is added to each well. After incubation for 25 to 30 min, the reaction is stopped with 100 μL of sulfuric acid (1 M H₂SO₄). The absorbance of the solution is measured at 450 nm with a microplate-reader spectrophotometer.

In contrast to the control, a positive colorimetric signal was obtained with the recombinant protein: the scFv does recognize the oLH.

The concentration of the periplasm was measured by a quantitative ELISA assay. 100 μl of a standard primary anti-oLH antibody (10-5-1-0.5-0.1-0.05-0.025 μg/ml) was deposited per well, on which oLH had been adsorbed beforehand at 1 μg/ml. After incubation of a peroxidase-coupled secondary antibody, and development with TMB, reading of the absorbance at 450 nm made it possible to obtain a standard range for estimating the concentration of scFv in the different dilutions of periplasms tested ranging from one half to ⅛th.

The concentration of the periplasm of clones 26/27 was thus estimated at 34 μg/μl.

In order to increase this concentration and remove the bacterial proteins, the scFv was purified starting from the anti-OX74 antibody coupled to Ademtech beads. The two elution fractions were combined into a single sample and after dialysis overnight at 4° C., their concentration was estimated by measuring the absorbance at 280 nm. The concentration measurements are presented in Table V below. The absorbance measured at 280 nm based on several dilutions made it possible to estimate the scFv concentration of the solution at 0.84 mg/ml.

TABLE V

| | OD | OD | Average | Estimated amount |
|---|---|---|---|---|
| OD 1 | 1/10th | 1/20th | OD | of protein |
| 1.211 | 0.106 | 0.068 | 1.21 | 0.84 mg/ml |

### EXAMPLE 5

### Investigation of the Potentiating Effect of the scFv on the Bioactivity of the oLH In Vitro

The potentiating effect of the scFv was characterized, on the bioactivity of the oLH and of a hormone that is homologous from the standpoint of activity, hCG. For this, bioassays were carried out in vitro on MLTC cells and in vivo in the rat (Example 6). The tests in vitro were carried out on MLTC cells (Mouse Leydig Tumor Cell) that stably express the LH receptor, and which, when stimulated with LH or hCG, secrete cAMP and progesterone (P4).

The response measured is the cAMP secretion after 3 hours of stimulation at 37° C. with an increasing range of LH alone or previously incubated with the periplasm or the purified scFv. The production of cAMP provides evidence of coupling of the LH receptor activated by the stimulating protein G (Gs) and therefore of signal transduction via the Gs/adenylate cyclase/PKA (cAMP-dependent protein kinase)/cAMP pathway. The response is expressed in picomoles of cAMP secreted per 100000 cells. Comparison of the biological response obtained in the presence of the hormone alone and that obtained with the hormone previously incubated with a supernatant makes it possible to measure whether the latter exerts a potentiating effect or no effect.

The MLTC cells were cultured in RPMI 1640 medium (with addition of L-glutamine and Hepes 25 mM) (Gibco, USA), which was supplemented with 10% of FBS (fetal bovine serum), 0.1% of gentamicin and with a mixture of penicillin and streptomycin according to the method described by Martinat et al. (Martinat et al., Reprod Nutr Dev., 45(1): 101-8, 2005). At 50% confluence, the cells are trypsinized and then seeded in 24-well plates at a rate of 100000 cells per well (300 μl) and left in a stove overnight. The cells are weaned next day for 2 hours at 37° C., 5% CO₂, replacing the medium in each well with 200 μl of weaning medium. The latter is identical to the growth medium but it has no FBS and contains 240 μM of IBMX (isobutylmethylxanthine), which is an inhibitor of phosphodiesterases. It prevents degradation of cAMP during stimulation, resulting in its accumulation, allowing proper evaluation of the efficacy of the agonist. In parallel, a range of oLH (0-5-10 and 20 ng/ml final) as well as different concentrations of the scFv to be tested (0.1 and 1 μg/ml) were prepared in a volume of 110 μl and preincubated for 1 h at 37° C. After weaning, the MLTC cells were stimulated with 50 μl of the various mixtures (scFv complexed or not with LH) and were put in the stove for 2 h. Each point of stimulation of LH alone or of the various complexes was tested in duplicate, on two culture wells. The supernatants were then recovered in glass tubes and the cAMP secreted following stimulation was measured using an ELISA kit according to the supplier's instructions (Biomedical Technologies, Inc., Stoughton, USA).

In parallel, the purified IgM B6 was also tested to see whether the potentiating effect that is observed with the scFv is greater or less than that obtained with the whole B6 antibody.

FIG. 7 illustrates the results obtained on stimulation with a range of oLH preincubated with scFv or IgM, purified and tested at a concentration of 0.1 μg/ml. The potentiating effect of the purified scFv is low and is not significant, in contrast to that of the IgM B6 (p<0.01). It can be seen that both the scFv alone, and the IgM alone, have no effect on cellular response when they are incubated without oLH (zero point of the curve).

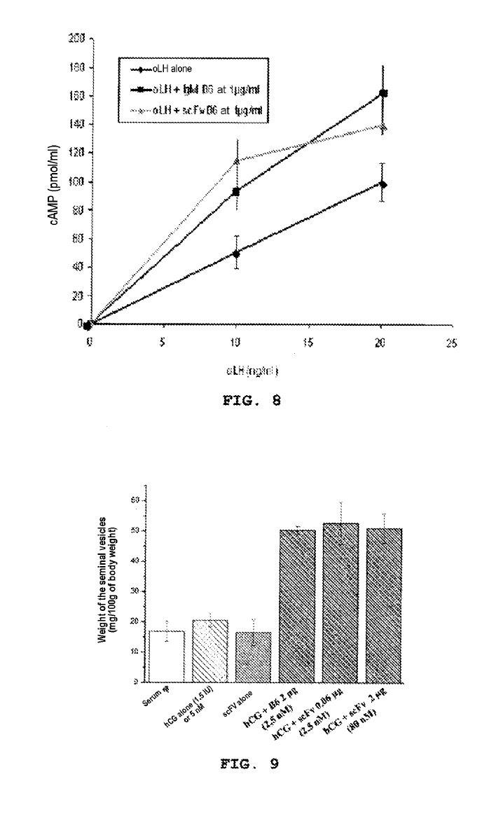

FIG. 8 illustrates the results obtained on stimulation with a range of oLH preincubated with the periplasm or the IgM at a concentration of 1 μg/ml. It can be seen that at this concentration, the potentiating effect of the scFv is very close to that of the IgM and is significant (p<0.05). The periplasm alone has no effect on the response (zero point of the curve).

### EXAMPLE 6

### Investigation of the Potentiating Effect of the scFv ON the Bioactivity of the OLH In Vivo, in the Rat

The objective was to verify that the effects observed in vitro on modulation of the biological activity of oLH by scFv can also be observed in vivo on the bioactivity of hCG, an analog of LH. To evaluate the potentiating effect of the scFv B6, a reference bioassay used by the pharmacopeia for determining the biological activity of LH or of hCG in the male rat is employed (Scobey et al., 2005, cited above). In this bioassay, the bioactivity of LH or of hCG is quantified relative to the increase in weight of the seminal vesicles, development of which is very androgen-dependent. Because of the very high cost of ovine LH, these bioassays were performed with hCG, which is readily available in a very pure form, and is inexpensive. In fact, hCG has a strict LH activity and is very well recognized by the potentiating antibody B6. It is regarded as an analog of LH.

The protocol was carried out with 25-day-old rats, which were injected with the hormone alone or previously incubated with the scFv or the antibody B6, once daily for 4 days, and then sacrificed on the 5th day to measure the weight of the seminal vesicles. The latter varies in proportion to the activity of hCG. Each condition was tested on a batch of 4 rats and was repeated in two independent experiments.

The samples of scFv and of IgM B6 were prepared in physiological saline solution at several concentrations. In previous experiments, the hCG/IgM B6 complex showed a maximum effect when IgM is injected at a concentration of 2.5 nM, or 2 μg. The weight of an IgM pentamer is 750 kDa whereas that of the scFv is estimated at 25 kDa. For comparing the potentiating effect of the IgM and of the scFv, the latter was tested at a concentration also of 2.5 nM, or 0.06 μg, so as to remain in equimolar conditions. The scFv was also tested at the same amount as the IgM, or 2 μg per injection.

These samples were preincubated or not with 1.5 IU of hCG at 37° C. for one hour. Each rat received 100 μl of the mixture per injection. On the fifth day, the rats were weighed and then sacrificed. Their seminal vesicles were removed and weighed. The weight of the seminal vesicles is expressed in mg/100 grams of body weight so as to be able to compare the results obtained with the different experimental batches.

FIG. 9 illustrates the effect of the complexes scFv/hCG and IgM/hCG on the weight of the seminal vesicles (n=1). When the scFv at 2 μg is injected alone, it does not produce any effect: the average weight of the seminal vesicles is at the same level as for the control batch that received an injection of physiological saline solution. When the scFv/hCG complex is tested at a concentration of 2 μg and 0.06 μg, it leads to a large increase (250%) in the weight of the seminal vesicles relative to injection of the hormone alone. The potentiating effect of the scFv at 2 μg reaches a level comparable to that of the IgM B6 injected at the same amount. However, when the scFv (0.06 μg) is tested in equimolar conditions, its potentiating effect is greater than that of the whole antibody.

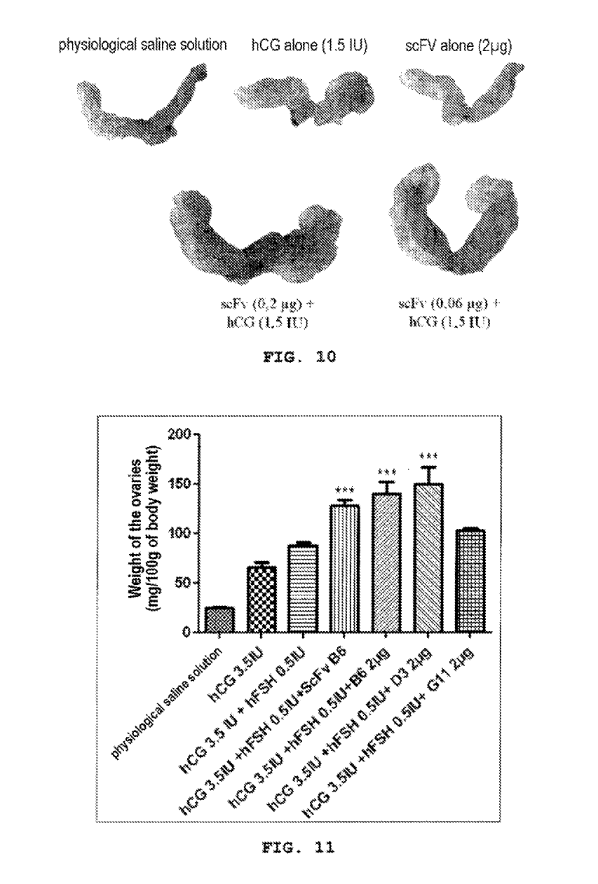

FIG. 10 illustrates the effect of the scFv/hCG complex on the size of the seminal vesicles. When the scFv (0.2 μg and 0.06 μg) is injected as a complex with hCG, it causes an increase in size of the seminal vesicles.

### EXAMPLE 7

### Potentiating Effect of the Mabs B6 and D3 as Well as of the scFv B60N the Activity of the oFSH and of the hFSH in the Female Rat

In order to verify that the FSH potentiating effect of the monoclonal antibodies B6 and D3, observed in vitro on LTK cells (Example 1), was indeed correlated with an FSH potentiating effect observed in vivo, their potentiating effect was tested on the bioactivity of the ovine and human FSH, in the female rat.

The protocol used for measuring FSH bioactivity is that of the bioassay described by Steelman and Pohley (Steelman S L, Pohley F M. Endocrinology, 53: 604-616, 1953), used as a reference protocol by the pharmacopeia.

Immature 21-day-old female rats receive 2 injections, morning and evening, of 100 μl of a mixture of hCG and FSH for three consecutive days. The injections are performed subcutaneously and comprise a constant amount of hCG (3.5 IU) with addition of a variable amount of FSH in the range from 0.5 to 1.5 IU of human FSH (Gonal F, Merck-Serono) or from 0.5 to 2 μg of ovine FSH. In order to quantify the potentiating effect of the MAb tested, other female rats are treated with the same mixture with addition of 2 μg of purified antibody, said mixture having been incubated beforehand for 20 min at 37° C.

On the fourth day, the rats are euthanased, weighed, and their ovaries are dissected and then weighed. The results are expressed in milligram of ovary/100 grams of body weight. The increase in weight of the ovaries is proportional to the amount of FSH injected, which makes it possible to quantify the bioactivity of the FSH injected as well as the potentiating effect of the MAb on the latter.

Their effect was evaluated on human FSH (Gonal F, recombinant hormone, Merck-Serono) used for stimulation of ovulation in women in the context of treatments for assisted conception (MAP). This hormone was selected on account of its high purity and its availability. In fact, it has already been shown that the MAbs B6 and D3 exert a potentiating effect on oFSH in vitro on LTK cells (Example 1). In contrast, the MAb G11 does not have a potentiating effect on oFSH in vitro on these same cells.

In this example, the MAbs B6 and D3 were tested in the form of whole antibodies, as well as in the form of scFv for B6 (scFv B6).

The G11 whole antibody was also investigated, as a control of the strict LH potentiating effect.

As shown in FIG. 11, a very significant potentiating effect (p<0.001) was obtained with B6, D3 and scFv B6 compared with the effect of the mixture hCG+hFSH injected without preincubation with the MAb. These results are the cumulative effect of two independent experiments and were conducted on 8 animals for each batch. Statistical analysis was performed with the GraphPad Prism software (GraphPad PRISM Software; GraphPad, San Diego, Calif.) by one-way analysis of variance and by the Bonferroni test (Bonferroni's Multiple Comparison Test).

However, G11 does not have a significant potentiating effect when it is injected with the mixture hCG+hFSH, which correlates well with its strict LH potentiating effect.

In the above experiments, as the female rats had been treated with a mixture of hCG and FSH, “control” experiments were conducted in order to differentiate and distinguish the potentiating effect exerted by these MAbs on the one hand on the activity of hCG and on the other hand on the activity of FSH. In fact, two control experiments were performed, consisting of injecting:

(1) FSH alone or preincubated with the MAb D3, or

(2) hCG alone or preincubated with the MAb D3.

The MAb D3 was selected for carrying out these experiments as it gives the largest FSH potentiating effect. The FSH used is hFSH Gonal F (Serono, Merck).

### “Control” Experiment (1):

The objective is to measure the potentiating effect of the MAbs on the FSH alone. The female rats were therefore treated with FSH alone or preincubated with the MAb.

The female rats were treated with (a) FSH at 0.5 IU, alone or preincubated with D3 (2 μg), (b) FSH at 1 IU, alone or preincubated with D3 (2 μg), or (c) FSH at 1.5 IU alone.

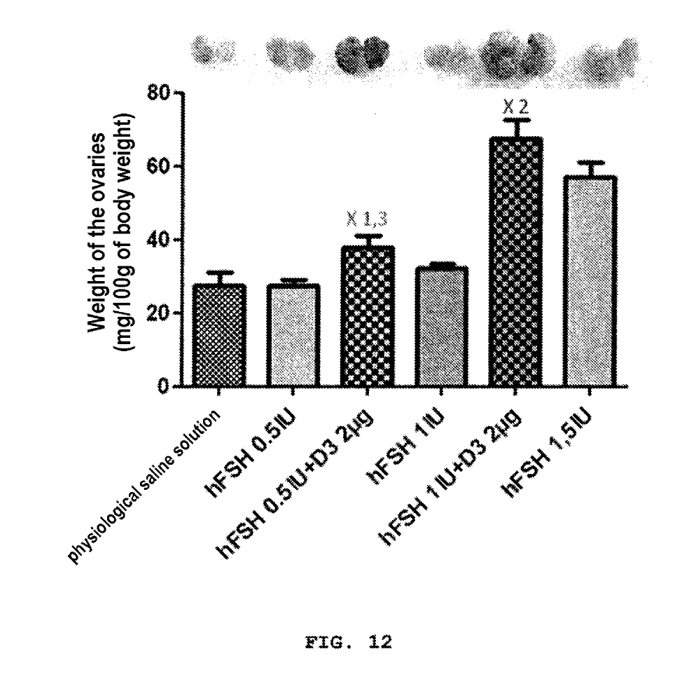

The results are shown in FIG. 12, in a diagram accompanied by corresponding photographs of the ovaries. The average weight of the ovaries from the batch treated with FSH+D3 is significantly higher than that of the batch treated with the hormone alone, particularly at a dose of 1 IU FSH, where a doubling of the weight of the ovaries is recorded with the complex. It should be emphasized that the ovarian stimulation observed with the batch FSH 1 IU+D3 is greater than that obtained with injection of 1.5 IU of FSH alone.

Comparison of the average weight of the ovaries in the rats treated with the hormone alone or with the FSH/D3 complex reveals a clear potentiating effect of the MAb, particularly with the dose 1 IU of FSH. In the latter case, the average weight of the ovaries is greater than that obtained in the rats treated with 1.5 IU FSH (n=5 rats per batch).

### “Control” Experiment (2):

The objective is to measure the potentiating effect of the MAb on hCG alone. For this, the female rats were treated with hCG alone or preincubated with the MAb according to the protocol of injections of the dosage of Steelman and Pooley.

The different batches treated are as follows:
hCG at 3.5 IU alone or preincubated with D3 (2 μg)
hFSH at 0.5 IU alone
hCG 3.5 IU+hFSH 0.5 IU
hCG 3.5 IU+hFSH 0.5 IU+D3 2 μg

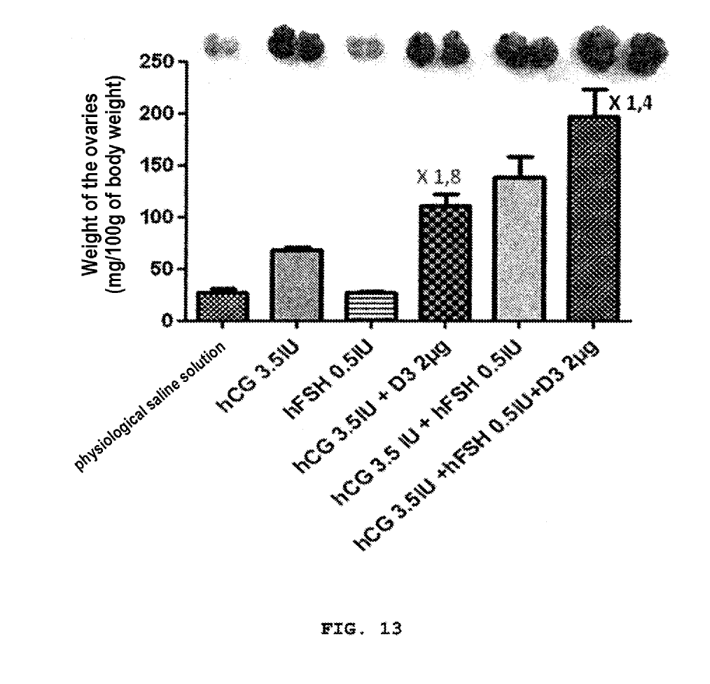

The results are shown in FIG. 13. They show that the average weight of the ovaries from the batch treated with hCG+D3 is greater than the average weight of the ovaries observed with the batch treated with hCG alone. There is therefore potentiation of hCG by the MAb, which is added to that exerted on FSH.

It should also be noted that the effect observed with the mixture MAb+hCG+hFSH leads to an increase in weight of the ovaries greater than the sum of the two separate effects: there is therefore a cooperative effect in the combination of the two treatments (hCG and FSH).

In conclusion, the effect observed with the mixture MAb+hCG+hFSH is indeed due to a potentiating effect of the MAb on FSH, independently of its potentiating effect on hCG. These “control” experiments also reinforce the demonstration of the potentiating effect of the MAbs D3 and B6 on the bioactivity of FSH.

These results demonstrate that the two MAbs, D3 and B6, exert a dual potentiating effect, on the activity of LH and on that of FSH.

### EXAMPLE 8

### Potentiating Effect of the mAb G11 In Vivo, in the Ile De France Ewe

This study was conducted on Ile de France ewes, pubescent, all of the same age. The objective was to evaluate, in vivo in the ewe, the potentiating effect of the MAb G11 on the activity of a porcine LH (pLH) injected to induce ovulation. The pLH, extracted from pig hypophyses, is used in certain treatments for inducing ovulation in ewes. For this, 3 mg of pLH is injected intravenously, 36 hours after sponge withdrawal.

Two protocols (A and B) were adopted. Their principle was to evaluate the potentiating effect of G11 on the activity of LH by dating on the one hand the moment of ovulation and, on the other hand, placement of the functional corpus luteum, reflected in an increase in progesterone secretion during the luteal phase. For this, the physiological parameters used were as follows:
number and dating of the ovulations deduced by endoscopic observation of the corpora lutea performed by laparoscopy, under anesthesia,
placement and monitoring of progesterone secretion by daily plasma analyses during the luteal phase.

The tests were conducted on the same pubescent Ile de France ewes, all of the same age (from 1 to 3 years). These females had all been synchronized prior to the protocols, by placement of a vaginal sponge impregnated with a progestagen (45 mg of flugestone acetate (FGA)—Intervet—France) for 14 days.

Protocol A: the potentiating effect of G11 was evaluated by injecting the pLH+MAb complex, previously incubated for 30 minutes at 37° C.:
placement of sponges for 14 days
intramuscular injection of the pLH+MAb complex, 36 hours after sponge withdrawal
endoscopy between 7 and 11 days after sponge withdrawal
daily collection of blood samples from the first day to the 21st day after sponge withdrawal, for analysis of plasma progesterone

Protocol B: the potentiating effect of G11 was evaluated by sequential injection of (1) pLH, and then (2) MAb 48 hours later:
placement of sponges for 14 days
intravenous injection of pLH alone (3 mg), 36 hours after sponge withdrawal
intramuscular injection of MAb alone (2 mg), 72 hours after sponge withdrawal, or 48 hours after the pLH
endoscopy 11 days after sponge withdrawal
daily collection of blood samples from the first day to the 21st day after sponge withdrawal, for analysis of plasma progesterone

### Results of Protocol A:

Two batches of 10 ewes received, by the intramuscular route, 36 hours after sponge withdrawal:
either 3 mg of pLH (batch with pLH alone)
or a mixture of 3 mg of pLH+2 mg of MAb G11 (batch pLH+MAb G11) incubated for 30 minutes at 37° C. prior to injection
   a—Endoscopic Analyses

An endoscopy was performed on each ewe in order to check whether ovulation has occurred and to date the corpus luteum or corpora lutea by the method described by Cognie J. et al. (Review Med. Vet. 2007, 158, 8-9, 447-451).

For each ewe, the results of the endoscopies made it possible, on the one hand, to precisely determine the number of ovulations (by counting the corpora lutea) and, on the other hand, to evaluate the moment of ovulation relative to sponge withdrawal (by dating the corpora lutea).

In the batch with pLH alone, all the ewes had ovulated and had a normal luteal phase.

In the batch pLH+MAb, all the ewes had ovulated. Just one had a short luteal phase (short cycle) with early regression of the corpus luteum. The other 9 had a normal luteal phase. This difference is not significant between the two batches.

The results of the ovulations are summarized in Table VI below.

TABLE VI

| | Batch with | Batch pLH + |
|---|---|---|
| | pLH alone | MAb G11 |
| Number of ewes without ovulation | 0 | 0 |
| Number of ewes with a regressed CL | 0 | 1 |
| and a short luteal phase | | |
| Number of ewes that have ovulated | 10 | 9 |
| and have a normal luteal phase | | |

As shown in Table VII below, the average moment of ovulation is 2.5 days after sponge withdrawal in batch pLH+MAb G11 versus 3.5 days in the batch with pLH alone. Statistical analysis by the T test indicates that this difference is significant at p<0.1 (GraphPad PRISM Software; GraphPad, San Diego, Calif.). No significant difference was observed in number of ovulations between the two batches.

TABLE VII

| | Batch pLH | Batch pLH + |
|---|---|---|
| | alone | MAb G11 |
| Average number of corpora | 2 ± 0.7 | 1.7 ± 1.06 |
| lutea per ewe | | |
| Average moment of ovulation | 3.5 ± 1.5 | 2.5 ± 0.81 |
| (in days after sponge withdrawal) | | |

Treatment with the MAb complexed with pLH therefore induces earlier ovulation with a shift of one day relative to treatment with pLH alone. This early initiation reflects a potentiating effect of the MAb G11 on LH activity.

b—Measurement of Progesterone Secretion (P4) During the Sexual Cycle

Blood samples were collected daily starting from the day of sponge withdrawal (D0) and up to the 21st day. Progesterone was determined by ELISA according to the protocol described by Canepa S. et al. (Cahiers Techniques INRA, 2008, 64, 19-30).

Only the ewes that had ovulated normally were taken into account; the one that had a short luteal phase (short cycle) was excluded.

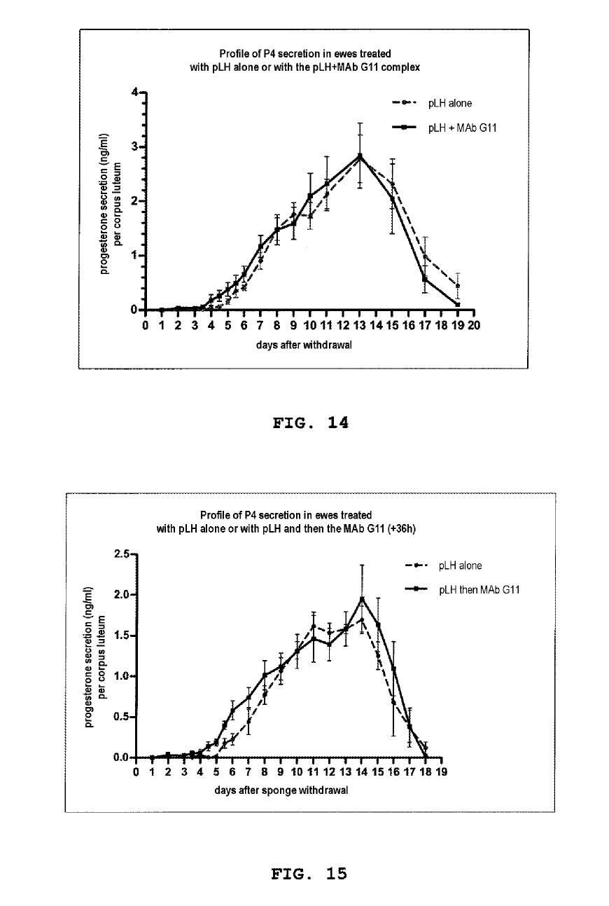

For each batch, the progesterone concentration values, measured at each date of the cycle, were averaged. So as to be able to average the concentrations of P4 of the females of one and the same batch, the baseline value of P4 measured on D1 after sponge withdrawal was regarded arbitrarily as the zero level of each female. Moreover, the results for P4 were expressed for one corpus luteum: in the cases where two corpora lutea were observed in a ewe, the value of P4 was divided by 2 for each measurement. The average curves obtained for each batch are presented in FIG. 14.

To compare the two complete curves, statistical analysis was performed by analysis of variance with two variables (two-way ANOVA) using the GraphPad Prism software (GraphPad PRISM Software; GraphPad, San Diego, Calif.). It shows that the two curves are not significantly different (p>0.1), which indicates that the pLH+MAb G11 complex injected intramuscularly does not induce a significant effect with this batch of 2×10 ewes. However, a trend toward a precocity of 12 to 24 hours in initiation of secretion of P4 is observed, signifying that, in the ewes in batch pLH+MAb G11, the corpus luteum becomes functional about 24 h earlier than in the ewes in the batch with pLH alone (4 days versus 5 days respectively). This shift is observed up to the eighth day after sponge withdrawal.

In batch pLH+MAb G11, the trend toward precocity of placement of a functional corpus luteum is correlated with a significant precocity of the moment of ovulation observed by endoscopy. In fact, a precocity of 24 hours is obtained in both cases, in favor of the batch pLH+MAb G11, for the two physiological events.

Taken together, these results indicate that the pLH+MAb G11 complex is capable of potentiating the activity of LH, in vivo, in the ewe. This potentiation is reflected in statistically earlier ovulation and a trend toward earlier induction of a steroidogenic response of the stimulated luteal cells in the ewes treated with the pLH+MAb G11 complex compared with the ewes treated with the hormone alone.

### Results of Protocol B:

The objective of protocol B was to evaluate whether the MAb injected separately from the hormone and with a time difference was also capable of exerting a potentiating effect on the circulating LH, in vivo, in the ewe.

For this, two batches of 8 ewes were treated, one with 3 mg of pLH alone, injected intravenously (IV) 36 hours after sponge withdrawal, and the other with 3 mg of pLH (intravenously), 36 hours after sponge withdrawal, then with 2 mg of the MAb G11 48 h later, by the intramuscular (IM) route.

a—Endoscopic Analyses

An endoscopy was performed on each ewe in order to check whether ovulation had occurred and to date the corpus luteum or corpora lutea by the method described by Cognie J. et al. (2007, cited above).

For each ewe, the results of the endoscopies made it possible, on the one hand, to precisely determine the number of ovulations, by counting the corpora lutea (CL), and, on the other hand, to evaluate the moment of ovulation relative to sponge withdrawal, by dating the corpora lutea.

Five females out of eight had ovulated normally in the batch pLH and six out of eight in the batch pLH/MAb. Two ewes had ovulated but had a short luteal phase.

The results of the ovulations are summarized in Table VIII below.

TABLE VIII

| | Batch | Batch |
|---|---|---|
| | pLH | pLH/MAb |
| Number of ewes without ovulation | 1 | 2 |
| Number of ewes with a regressed CL | 2 | 0 |
| and with a short luteal phase | | |
| Number of ewes that have ovulated | 5 | 6 |
| and have a normal luteal phase | | |

As shown in Table IX below, the average moment of ovulation is 2.83 days after sponge withdrawal in the batch pLH/MAb G11 versus 3.71 days in the batch with pLH alone. Statistical analysis by the T test indicates that this difference is significant at p<0.1 (GraphPad PRISM Software; GraphPad, San Diego, Calif.). No difference was observed in number of ovulations.

TABLE IX

| | Batch | Batch |
|---|---|---|
| | pLH | pLH/MAb |
| Average number of corpora | 1.14 ± 0.37 | 1.16 ± 0.4 |
| lutea per ewe | | |
| Average moment of ovulation | 3.71 ± 1.11 | 2.83 ± 1.16 |
| (in days after sponge withdrawal) | | |

b—Measurement of Progesterone Secretion (P4) During the Sexual Cycle

Blood samples were collected daily starting from the day of sponge withdrawal (D0) and up to the 21st day. Progesterone was determined by ELISA according to the protocol described by Canepa S. et al. (2008, cited above).

Only the ewes that had ovulated normally were considered; those that had a short luteal phase (short cycle) were excluded.

For each batch, the progesterone concentration values, measured at each date of the cycle, were averaged. So as to be able to average the concentrations of P4 of the females in one and the same batch, the baseline value of P4 measured on D1 after sponge withdrawal was regarded arbitrarily as the zero level of each female. Moreover, the results for P4 were expressed for one corpus luteum: in the cases where two corpora lutea were observed in a ewe, the value of P4 was divided by 2 for each measurement. The average curves obtained for each batch are presented in FIG. 15.

The results were compiled and analyzed statistically with the GraphPad Prism software (GraphPad PRISM Software; GraphPad, San Diego, Calif.). To compare the two complete curves, statistical analysis was performed by analysis of variance with two variables (two-way ANOVA). It shows that the two curves are significantly different (p<0.05), which indicates that the MAb G11, injected alone, exerts a potentiating effect, significant at p<0.05, on the activity of LH.

This potentiating effect is manifested by a precocity of 24 hours in the initiation of secretion of P4 (4.5 days after withdrawal on average) relative to the batch with pLH alone, where secretion is initiated 5.5 days after sponge withdrawal. These results signify that, in the ewes in the batch pLH/MAb, the corpus luteum becomes functional 24 h earlier than in the ewes in the batch with pLH alone. This difference is clearly observed up to the ninth day after sponge withdrawal.

In the batch pLH/MAb G11, precocity of placement of a functional corpus luteum is correlated with precocity of the moment of ovulation observed by endoscopy. In fact, in both cases a gap of about 24 hours is obtained between the two batches, each time with a precocity of the two physiological events in the batch treated with the MAb.

Taken together, these results indicate that the MAb injected alone, by the intramuscular route, is capable of binding to the ovine LH present in the blood circulation and of potentiating its activity. This potentiation is reflected in quicker induction of the steroidogenic response of the luteal cells stimulated by the plasma LH/MAb complex.

It should also be pointed out that as the half-life of the pLH is very short (20 min in the ewe), this hormone had been eliminated completely at the moment of injection of the MAb, which was delayed by 48 hours relative to that of the pLH. This means that the results obtained are due to the potentiating effect of the MAb G11 on the animal's endogenous LH. They therefore demonstrate that, in a large animal, in this case the ewe, the MAb injected alone can complex with the endogenous LH and induce potentiation of its effect, in vivo.

### EXAMPLE 9

### Characterization of the Specificity of the Mabs B6, D3 and G11

The specificity of the MAbs was investigated by ELISA. Each hormone evaluated was adsorbed, for 18 h at 4° C., on the wells of an ELISA plate at a concentration of 2 μg/ml in 0.1 M sodium carbonate buffer at a rate of 100 μl per well.

After washing five times (with PBS—Tween 0.1%) and a surcoating step (100 μl of PBS—Tween 0.1%—BSA 1%, 45 min at 37° C.), each MAb, prepared at concentrations of 0.1-1 and 10 μg/ml, was incubated for 1 hour at 37° C.

After washing five times, a secondary antibody (peroxidase-coupled mouse anti-IgM, Jackson Laboratories) was incubated for 1 h at 37° C. (100 μl/well). After washing five times, the peroxidase is developed with TMB (100 μl/well), for 30 min at room temperature and then stopped with 1M H₂SO₄ (50 μl/well).

The intensity of the color reaction is quantified (OD) and will serve as a reference for calculating the percentage of cross reaction for each hormone tested. Both for the LHs and for the FSHs, the value of OD measured with the reference ovine hormone is regarded as the 100% value. The percentage of cross reaction is the ratio of OD hormone tested to OD ovine hormone×100.

1) Cross Reaction with the LHs of Porcine and Bovine Origin and with Human Chorionic Gonadotropin (hCG)

The three MAbs recognize the porcine and bovine LHs with a percentage of cross reaction greater than or equal to the oLH, and also cross with the human hormone hCG (see Table X below).

TABLE X

| | B6 | D3 | G11 |
|---|---|---|---|
| oLH | 100% | 100% | 100% |
| pLH | 168% | 170% | 140% |
| bLH | 122% | 121% | 100% |
| hCG | 97% | 72% | 68% |

2) Cross Reaction with the FSHs of Porcine, Bovine and Human Origin

The three MAbs recognize the porcine, bovine and human FSHs with a percentage of cross reaction greater than or equal to the oFSH (see Table XI below).

TABLE XI

| | B6 | D3 | G11 |
|---|---|---|---|
| oFSH | 100% | 100% | 100% |
| pFSH | 137% | 100% | 230% |
| bFSH | 430% | 460% | 380% |
| hFSH | 100% | 100% | 113% |

The three MAbs therefore have a similar specificity profile, characterized by a broad spectrum of recognition in favor of the porcine, bovine and human homologous hormones.

3) Cross Reaction with Equine Choriogonadotropin (eCG) or PMSG

In the same way, the antibodies were evaluated on the commercial eCG (Synchro Part, CEVA, Libourne, France) used in treatments for induction of ovulation in sheep and goats. An isotypic control (IgM directed against another type of antigen very remote from the gonadotropic hormones). Each antibody was incubated at a concentration of 10 μg/ml and 1 μg/ml. The results, expressed in units of optical density (OD), are presented in Table XII below.

TABLE XII

| OD on | | | | Control |
|---|---|---|---|---|
| commercial eCG | B6 | D3 | G11 | antibody |
| Antibody at 10 μg/ml | 0.088 | 0.091 | 0.09 | 0.09 |
| Antibody at 1 μg/ml | 0.078 | 0.102 | 0.088 | 0.09 |

No cross reaction with the commercial eCG is observed: the value of OD is the same whether with the specific antibodies or with the isotypic control.

The same results were obtained on evaluating the antibodies on purified eCG (6000 IU/mg).

### EXAMPLE 10

### Polymorphism of the Frameworks 10F the Mabs B6, D3 and G11

The three MAbs B6, D3 and G11 have identical sequences of CDR1, CDR2 and CDR3 for their light chain and for their heavy chain.

Moreover, the sequences of the frameworks FR2, FR3 and FR4 of their VH and VL chains are identical.

Only the FR1 sequences of VH and VL vary depending on the MAb.

### 1) FR1 of the Light Chain (VL):

A polymorphism is observed in the residues in position 4 and 7 of FR1 of the light chain (see Table XIII below).

TABLE XIII

| | FR1 (amino acids 1 to 10 of VL) | |
|---|---|---|
| B6 | DIVMTQATSS | (SEQ ID NO: 20) |
| D3 | ---KTQTTSS | (SEQ ID NO: 22) |
| G11 | DIQMTQTTSS | (SEQ ID NO: 18) |

In position 4, it is noted that there is a methionine (M), amino acid with nonpolar hydrophobic side chain, or a lysine (K), amino acid with positively charged side chain. This polymorphism induces a large change in physicochemical properties, owing to the presence or absence of a positive charge, but does not constitute a structural element common to the two MAbs potentiating the bioactivity of LH and of FSH.

In position 7, it is noted that there is an alanine (A), amino acid with hydrophobic nonpolar chain, or a threonine (T), amino acid with uncharged polar chain. In this case it is a polymorphism that is relatively conservative of the physicochemical properties of the amino acids. Once again, this does not constitute a structural element common to the two MAbs potentiating the bioactivity of LH and of FSH.

Therefore, in the light chain, no polymorphism of FR1 appears to be associated with the dual LH and FSH potentiating effect.

### 2) FR1 of the Heavy Chain (VII):

A polymorphism is observed at the level of the residues in position 3 and 6 of FR1 of the heavy chain (see Table XIV below).

TABLE XIV

| | FR1 (amino acids 1 to 10 of VH) | |
|---|---|---|
| B6 | EVQLQQSGAE | (SEQ ID NO: 21) |
| D3 | EVQLQESGAE | (SEQ ID NO: 23) |
| G11 | EVKLQQSGAE | (SEQ ID NO: 19) |

In position 3, it is noted that there is a glutamine (Q), amino acid with uncharged polar chain, or a lysine (K), amino acid with positively charged side chain. This time it is a polymorphism inducing a large change in the physicochemical properties of this region. The presence of a glutamine in position 3 appears to be specific to the two MAbs potentiating the bioactivity of LH and of FSH.

In position 6, it is noted that there is a glutamine (Q), amino acid with uncharged polar chain, or a glutamic acid (E), amino acid with negatively charged side chain. Once again, it is a polymorphism inducing a large change in physicochemical properties, but which in this case does not appear to be associated with the dual LH and FSH potentiating effect.

### EXAMPLE 11

### Effect of the Antibody D3 and of the scFv B60N Progesterone Production by the MLTC Cells Stimulated by oLH or hCG

The cells are cultured in RPMI 1640 medium supplemented with 10% FBS and 1% penicillin/streptomycin. They are weaned 1 hour before stimulation with the hormone (oLH or hCG) alone or the antibody/hormone or scFv/hormone complex.

Stimulation with oLH:

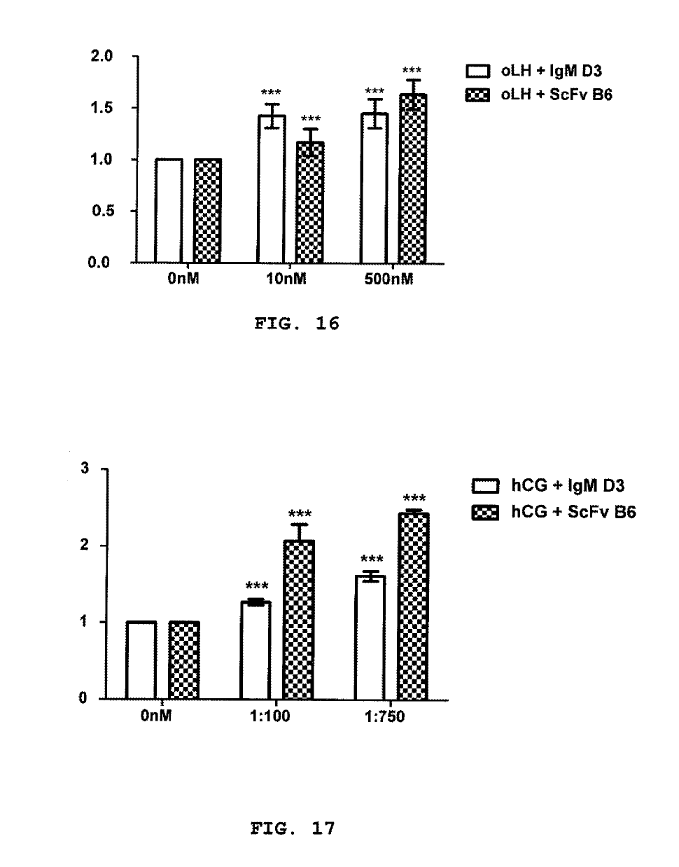

The amount of progesterone secreted by the MLTC cells stimulated with 0.5 nM of oLH alone was compared with that secreted by the MLTC cells stimulated with 0.5 nM of oLH preincubated with the complete IgM D3 or scFv B6, at 10 nM or 500 nM. The results are presented in FIG. 16. The abscissa shows the concentrations of IgM or scFv (0 nM for stimulation with oLH alone); on the ordinate, the ratio of the amount of progesterone secreted in the presence of the D3/oLH or B6/oLH complex to the amount of progesterone secreted in the presence of oLH alone. At both concentrations, scFv B6 and IgM D3 exert a significant potentiating effect on the steroidogenic response of the MLTC cells, relative to stimulation with oLH alone (p<0.001 by the Bonferroni test). The potentiating effect is maximal starting from a concentration of 10 nM of IgM D3 and a concentration of 500 nM for scFv B6.

Stimulation with hCG:

The same experiment was carried out with hCG at a constant concentration of 0.05 nM. The amount of progesterone secreted was compared in the case of stimulation with 0.05 nM of hCG alone and in the case of stimulation with the complex hCG 0.05 nM+complete IgM D3 or hCG 0.05 nM+scFv B6, at 5 nM and 37.5 nM. The results are presented in FIG. 17. The abscissa shows the concentrations of IgM or scFv (0 nM for stimulation with hCG alone); on the ordinate, the ratio of the amount of progesterone secreted in the presence of the D3/hCG or scFv B6/hCG complex to the amount of progesterone secreted in the presence of hCG alone. At both concentrations, scFv B6 and IgM D3 exert a significant potentiating effect on the steroidogenic response of the MLTC cells, relative to stimulation with hCG alone (p<0.001 by the Bonferroni test). The potentiating effect is maximal at a concentration of 37.5 nM of IgM D3 or of scFv B6.

### EXAMPLE 12

### Potentiating Effect In Vivo of a Diabody Derived from the B6 B5P0 Antibody on FSH Activity in the Female Rat

A diabody, designated B5P0 hereinafter, was constructed from the sequence of the VH and VL of the antibody B6, joined by a linker with 5 amino acids.

The nucleotide and peptide sequences of this diabody are shown in the appended sequence listing under numbers SEQ ID NO: 29 and SEQ ID NO: 30, respectively.

The potentiating effect of the diabody B5P0 on FSH activity in vivo was determined by the Steelman and Pohley test in the immature female rat, as described in Example 7 above.

The results are shown in FIG. 18.

These results show that the diabody B5P0 (2 μg) previously incubated with 0.5 IU of hFSH exerts a potentiating effect equivalent to that of the complete IgM B6 (2 μg). This effect leads in both cases to an increase in the weight of the ovaries by a factor of 2.1, which is highly significant (p<0.001, Bonferroni test).

### EXAMPLE 13

### Potentiating Effect of the scFv B6 In Vivo on the LH and FSH Activities in the Ewe

a) Potentiating Effect of the scFv B6 on LH Activity

The study was conducted on pubescent Ile de France ewes, aged 3 years. The potentiating effect of the scFv B6 on the activity of the endogenous LH was evaluated in comparison with that of the complete IgM G11.

The ewes had all been synchronized by placement of a vaginal sponge impregnated with a progestagen (45 mg of flugestone acetate (FGA)—Intervet—France) for 14 days.

36 hours after sponge withdrawal, the animals received an injection of 3 mg of pLH intravenously. The ewes were divided into three batches, A, B, and C:
batch A: treated with pLH alone
batch B: treated with pLH and then scFv B6;
batch C: treated with pLH and then IgM G11.

72 hours after sponge withdrawal, the animals in batches B and C received, respectively, 2 mg of purified scFv B6 or 2 mg of purified IgM G11, by the intramuscular route.

Blood samples are collected daily from the first day to the 8th day after sponge withdrawal for analysis of plasma progesterone. Eight days after sponge withdrawal, endoscopies are carried out for counting and dating the corpora lutea.

The endoscopy results are presented in Table XV.

TABLE XV

| | Number of | Dating of the |
|---|---|---|
| Treatment | corpora lutea | corpora lutea |
| Batch A: pLH alone 3 mg | 2.09 ± 3.23 | 4.06 ± 4.7 |
| Batch B: pLH then scFv B6P 2 mg | 1.33 ± 0.58 | 5 ± 0.5 |
| Batch C: pLH then IgM G11 2 mg | 2 ± 0.71 | 5.2 ± 0.27 |

The number of corpora lutea is expressed as mean±standard deviation. There is no significant difference between the number of corpora lutea obtained in the three batches [analysis by T test (GraphPad PRISM Software; GraphPad, San Diego, Calif.)]. The dating of the corpora lutea is expressed as the number of days post-ovulation (mean±standard deviation). The average age of the corpora lutea is 5 days for the batches treated with IgM G11 or scFv B6P versus an average age of 4 days for the batch treated with pLH alone. This difference in age of the corpora lutea between batch A and batches B and C is significant (p<0.05). There is no significant difference between batches B and C. These results mean that in the ewes treated with pLH and then IgM or scFv, ovulation took place 1 day before that observed in the ewes treated with pLH alone. The scFv B6 therefore exerts the same potentiating effect in vivo in the ewe, as the whole antibody G11.

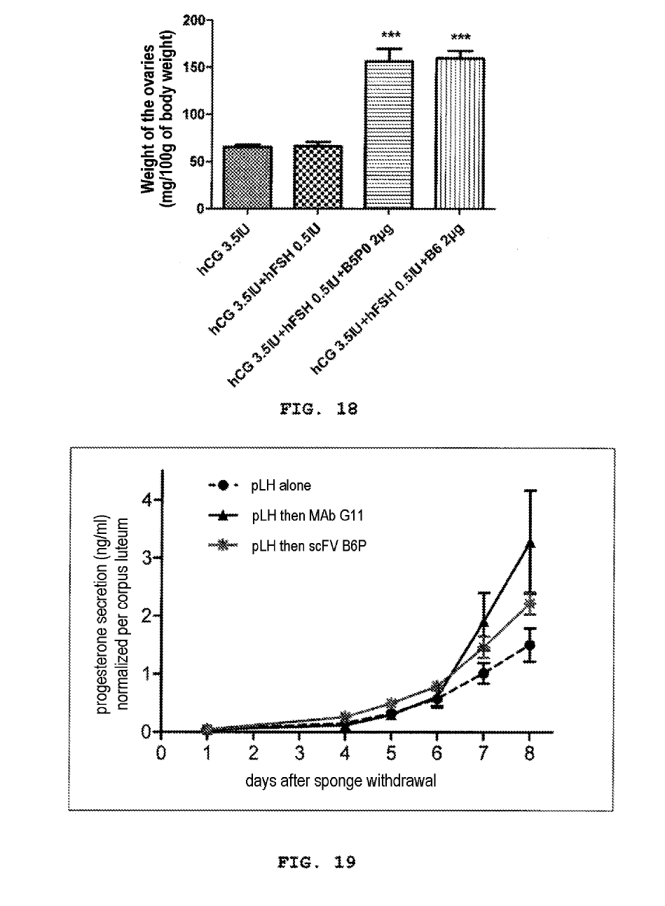

The profile of progesterone secretion at the start of the luteal phase is shown in FIG. 19.

For each batch, the progesterone concentration values (ng/ml) were normalized per number of corpora lutea. Each curve represents the mean of the progesterone values obtained in the females in each batch. The profiles of P4 secretion were compared by analysis of variances with two variables (two-way ANOVA, GraphPad PRISM Software; GraphPad, San Diego, Calif.). This analysis showed that the curve of average P4 secretion is significantly different between batch A and batches B and C (p>0.05) and that there is no difference between the curves for batch B and C. These results indicate that the scFv B6, like the MAb G11, injected alone, exerts a potentiating effect, manifested by development of progesterone secretion that is greater and quicker than in the batch treated with pLH alone. This result is correlated with the precocity of the moment of ovulation observed by endoscopy in the ewes treated with pLH and then scFv B6P or G11.

Taken together, these results indicate that the scFv B6 is capable of binding to endogenous ovine LH, and of potentiating its activity in vivo, with the same efficacy as the complete MAb G11.

b) Potentiating Effect of the scFv B6 on FSH Activity

The study was conducted in the sexual rest period (long days) and related to 18 ewes aged 4 years. The ewes had all been synchronized prior to the protocols, by placement of a vaginal sponge impregnated with a progestagen (45 mg of flugestone acetate (FGA)—Intervet—France) for 14 days.

24 hours and 12 hours before sponge withdrawal, the ewes received intramuscular injections of 100 μg and then of 83 μg of pure pFSH.

The ewe were divided into two batches:
batch A: treated with pFSH alone
batch B: treated with pFSH and then scFv B6.

The ewes in batch B received, by the intramuscular route, 3 successive injections of 1 mg of purified scFv B6P: the first on D0 during sponge withdrawal, the second on D1, and the third on D3.

On D7: endoscopies were performed for counting the number of corpora lutea.

The preovulatory peak of LH was measured by quantitative ELISA assay for all the females.

The endoscopy results and assay of LH are presented in Table XVI:

TABLE XVI

| | | Number of | Dating of the |
|---|---|---|---|
| | Number of | corpora lutea in | LH peak in hours |
| | ewes that | the ewes that | after sponge |
| Treatment | had ovulated | had ovulated | withdrawal |
| Batch A: pFSH alone | 1/7 | 6 | 54 |
| Batch B: pFSH then | 3/7 | 23 | 48 |
| scFv B6P | | 16 | 48 |
| | | 3 | 48 |

It can be seen that the number of females that had ovulated is significantly higher in the batch treated with pFSH and then scFv B6P (3/7 versus 1/7). Batch B also shows a significantly higher number of ovulations [(p<0.001), analysis by T test (GraphPad PRISM Software; GraphPad, San Diego, Calif.)]. Moreover, in the batch pFSH then scFv B6P, the three females that had ovulated had a synchronized LH peak, at 48 hours after sponge withdrawal, which was advanced by 12 hours relative to that of the batch with pFSH alone. This difference is significant (p<0.005).

These results indicate that, taking into account the short half-life of pFSH (30 minutes), the scFv B6P induced a potentiating effect on endogenous FSH, reflected in a higher number of ovulations and a very synchronized LH peak.

1. A ligand of a luteinizing hormone (LH), characterized in that it comprises the paratope of an anti-ovine LH antibody the heavy chain variable domain of which contains the following CDRs:
(SEQ ID NO: 13)
VH-CDR1, defined by the sequence GYTFTNYW;
(SEQ ID NO: 14)
VH-CDR2, defined by the sequence IYPGGGYT;
(SEQ ID NO: 15)
VH-CDR3, defined by the sequence
ARTPLYGSSYGGFAY;
and the light chain variable domain of which contains the following CDRs:
(SEQ ID NO: 16)
VL-CDR1, defined by the sequence QGISNY;
VL-CDR2, defined by the sequence YTS;
(SEQ ID NO: 17)
VL-CDR3, defined by the sequence QQYSKLPWT.

2. The ligand as claimed in claim 1, wherein the heavy chain contains a framework region FR1 characterized in that the N-terminal portion of the framework region FR1 of the heavy chain is defined by the sequence X₁VQLQX₁SGAE (SEQ ID NO: 24) in which X₁ represents a glutamine or a glutamic acid.

3. The ligand as claimed in claim 2, wherein the heavy chain contains a framework region FR1 characterized in that the N-terminal portion of the framework region FR1 of the heavy chain is defined by the sequence SEQ ID NO: 24 in which X₁ represents a glutamine.

4. The ligand as claimed in claim 2, wherein the light chain contains a framework region FR1 characterized in that the N-terminal portion of the framework region FR1 of the light chain contains the sequence X₂TQX₃TSS (SEQ ID NO: 25), in which X₂ represents a methionine or a lysine and X₃ represents a threonine or an alanine.

5. The ligand as claimed in claim 2, selected from:
a) the monoclonal antibody 1A6 C4 G11 produced by the hybridoma CNCM I-4332;
b) a Fab, Fab′, or Fab′2 fragment of an antibody a), above; or
c) a recombinant protein comprising the paratope of an antibody a) above.

6. The ligand as claimed in claim 3, selected from:
a) the monoclonal antibody 9A4 A7 D3 produced by the hybridoma CNCM I-4333;
b) the monoclonal antibody 9A4 D4 B6 produced by the hybridoma CNCM I-4334;
c) a Fab, Fab′, or Fab′2 fragment of an antibody a) or b) above; or
d) a recombinant protein comprising the paratope of an antibody a) or b) above.

7. The ligand as claimed in claim 1, for use as a medicinal product.

8. A method for potentiating the bioactivity of LH which comprises contacting a ligand as claimed in claim 1 with LH.

9. A method for potentiating the bioactivity of LH and the bioactivity of follicle-stimulating hormone (FSH) which comprises contacting a ligand as claimed in claim 1 with LH or FSH.

10. A ligand-gonadotropin complex selected from:
a complex of a ligand as claimed in claim 1 with LH or hCG; or
a complex of a ligand as claimed in claim 1 with FSH.

11. The complex as claimed in claim 10, for use as a medicinal product.

12. A method for inducing ovulation in a female mammal which comprises administering to said female mammal a ligand as claimed in claim 1, a complex thereof with LH or hCG; or a complex thereof with FSH.

13. A method for treating a pathological state in a subject resulting from low circulating levels of LH and FSH which comprises administration to the subject of a ligand of claim 1.

14. The method of claim 13 wherein the pathological state is a disorder for resulting from hypophyseal insufficiency.

15. A method for treating a male or female subject for hyporeceptivity of the gonads to LH and FSH which comprises administration to the subject of a ligand of claim 1.

16. The ligand as claimed in claim 3, wherein the light chain contains a framework region FR1 characterized in that the N-terminal portion of the framework region FR1 of the light chain contains the sequence X₂TQX₃TSS (SEQ ID NO: 25), in which X₂ represents a methionine or a lysine and X₃ represents a threonine or an alanine.

17. A scFv fragment of sequence SEQ ID NO: 28.


SEQUENCE LISTING (30 sequences)

SEQ ID NO: 1 - DNA, 368 bp, Mus musculus
gaggtccaactgcaggagtcaggagctgagctggtaaggcctgggacttcagtgaagatatcctgcaaggcttctggctacaccttcactaactactggctaggttgggtaaagcagaggcctggacatggacttgagtggattggagatatttaccctggaggtggttatactaactacaatgagaagttcaagggcaaggccacactgactgcagacacatcctccagcactgcctacatgcagctcagtagcctgacatctgaggactctgctgtctatttctgtgcaagaacccctctctacggtagtagctacggggggtttgcttactggggccaagggactctggtcactgtctctgcaga

SEQ ID NO: 2 - protein, 122 aa, Mus musculus
  MISC_FEATURE (26)..(33): residues constituting the VH-CDR1
EVQLQESGAELVRPGTSVKISCKASGYTFTNYWLGWVKQRPGHGLEWIGDIYPGGGYTNYNEKFKGKATLTADTSSSTAYMQLSSLTSEDSAVYFCARTPLYGSSYGGFAYWGQGTLVTVSA

SEQ ID NO: 3 - DNA, 313 bp, Mus musculus
aagacacagactacatcctccctgtctgcctctctgggagacagagtcaccatcagttgcagtgcaagtcagggcattagcaattatttaaactggtatcagcagaaaccagatggaactgttaaactcctgatctattacacatcaagtttacactcaggagtcccatcaaggttcagtggcagtgggtctgggacagattattctctcaccatcagcaacctggaacctgaagatattgccacttactattgtcagcagtatagtaagcttccgtggacgttcggtggaggcaccaagctggaaatcaaac

SEQ ID NO: 4 - protein, 104 aa, Mus musculus
  MISC_FEATURE (24)..(29): residues constituting the VL-CDR1
KTQTTSSLSASLGDRVTISCSASQGISNYLNWYQQKPDGTVKLLIYYTSSLHSGVPSRFSGSGSGTDYSLTISNLEPEDIATYYCQQYSKLPWTFGGGTKLEIK

SEQ ID NO: 5 - DNA, 368 bp, Mus musculus
gaggtgaagctgcagcagtcaggagctgagctggtaaggcctgggacttcagtgaagatatcctgcaaggcttctggctacaccttcactaactactggctaggttgggtaaagcagaggcctggacatggacttgagtggattggagatatttaccctggaggtggttatactaactacaatgagaagttcaagggcaaggccacactgactgcagacacatcctccagcactgcctacatgcagctcagtagcctgacatctgaggactctgctgtctatttctgtgcaagaacccctctctacggtagtagctacggggggtttgcttactggggccaagggactctggtcactgtctctgcaga

SEQ ID NO: 6 - protein, 122 aa, Mus musculus
  MISC_FEATURE (26)..(33): residues constituting the VH-CDR1
EVKLQQSGAELVRPGTSVKISCKASGYTFTNYWLGWVKQRPGHGLEWIGDIYPGGGYTNYNEKFKGKATLTADTSSSTAYMQLSSLTSEDSAVYFCARTPLYGSSYGGFAYWGQGTLVTVSA

SEQ ID NO: 7 - DNA, 322 bp, Mus musculus
gatatccagatgacacagactacatcctccctgtctgcctctctgggagacagagtcaccatcagttgcagtgcaagtcagggcattagcaattatttaaactggtatcagcagaaaccagatggaactgttaaactcctgatctattacacatcaagtttacactcaggagtcccatcaaggttcagtggcagtgggtctgggacagattattctctcaccatcagcaacctggaacctgaagatattgccacttactattgtcagcagtatagtaagcttccgtggacgttcggtggaggcaccaagctggaaatcaaac

SEQ ID NO: 8 - protein, 107 aa, Mus musculus
  MISC_FEATURE (27)..(32): residues constituting the VL-CDR1
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPWTFGGGTKLEIK

SEQ ID NO: 9 - DNA, 366 bp, Mus musculus
gaggtgcaactgcagcagtctggagctgagctggtaaggcctgggacttcagtgaagatatcctgcaaggcttctggctacaccttcactaactactggctaggttgggtaaagcagaggcctggacatggacttgagtggattggagatatttaccctggaggtggttatactaactacaatgagaagttcaagggcaaggccacactgactgcagacacatcctccagcactgcctacatgcagctcagtagcctgacatctgaggactctgctgtctatttctgtgcaagaacccctctctacggtagtagctacggggggtttgcttactggggccaagggactctggtcactgtctctgca

SEQ ID NO: 10 - protein, 122 aa, Mus musculus
  MISC_FEATURE (26)..(33): residues constituting the VH-CDR1
EVQLQQSGAELVRPGTSVKISCKASGYTFTNYWLGWVKQRPGHGLEWIGDIYPGGGYTNYNEKFKGKATLTADTSSSTAYMQLSSLTSEDSAVYFCARTPLYGSSYGGFAYWGQGTLVTVSA

SEQ ID NO: 11 - DNA, 321 bp, Mus musculus
gatattgtgatgacgcaggctacatcctccctgtctgcctctctgggagacagagtcaccatcagttgcagtgcaagtcagggcattagcaattatttaaactggtatcagcagaaaccagatggaactgttaaactcctgatctattacacatcaagtttacactcaggagtcccatcaaggttcagtggcagtgggtctgggacagattattctctcaccatcagcaacctggaacctgaagatattgccacttactattgtcagcagtatagtaagcttccgtggacgttcggtggaggcaccaagctggaaatcaaa

SEQ ID NO: 12 - protein, 107 aa, Mus musculus
  MISC_FEATURE (27)..(32): residues constituting the VL-CDR1
DIVMTQATSSLSASLGDRVTISCSASQGISNYLNWYQQKPDGTVKLLIYYTSSLHSGVPSRFSGSGSGTDYSLTISNLEPEDIATYYCQQYSKLPWTFGGGTKLEIK

SEQ ID NO: 13 - protein, 8 aa, Mus musculus
GYTFTNYW

SEQ ID NO: 14 - protein, 8 aa, Mus musculus
IYPGGGYT

SEQ ID NO: 15 - protein, 15 aa, Mus musculus
ARTPLYGSSYGGFAY

SEQ ID NO: 16 - protein, 6 aa, Mus musculus
QGISNY

SEQ ID NO: 17 - protein, 9 aa, Mus musculus
QQYSKLPWT

SEQ ID NO: 18 - protein, 10 aa, Mus musculus
DIQMTQTTSS

SEQ ID NO: 19 - protein, 10 aa, Mus musculus
EVKLQQSGAE

SEQ ID NO: 20 - protein, 10 aa, Mus musculus
DIVMTQATSS

SEQ ID NO: 21 - protein, 10 aa, Mus musculus
EVQLQQSGAE

SEQ ID NO: 22 - protein, 7 aa, Mus musculus
KTQTTSS

SEQ ID NO: 23 - protein, 10 aa, Mus musculus
EVQLQESGAE

SEQ ID NO: 24 - protein, 10 aa, Artificial Sequence
  Consensus Sequence
XVQLQXSGAE

SEQ ID NO: 25 - protein, 7 aa, Artificial Sequence
  Consensus Sequence
XTQXTSS

SEQ ID NO: 26 - protein, 10 aa, Artificial Sequence
  Consensus Sequence
DIXXTQXTSS

SEQ ID NO: 27 - protein, 10 aa, Artificial Sequence
  Consensus Sequence
DIQMTQATSS

SEQ ID NO: 28 - protein, 244 aa, artificial sequence
  scFv B6
QVQLQQSGAELVRPGTSVKISCKASGYTFTNYWLGWVKQRPGHGLEWIGDIYPGGGYTNYNEKFKGKATLTADTSSSTAYMQLSSLTSEDSAVYFCARTPLYGSSYGGFAYWGQGTLVTVSAGGGGSGGGGSGGGGSDIQMTQTXSSLSASLGDRVTISCSASQGISNYLNWYQQKPDGTVKLLIYYTSSLHSGVPSRFSGSGSGTDYSLTISNLEPEDIATYYCQQYSKLPWTFGGGTKLEIK

SEQ ID NO: 29 - DNA, 711 bp, Artificial sequence
  Diabody
caggttcagctgcagcagagcggtgcagaactggttcgtccgggtacaagcgttaaaattagctgtaaagccagcggctatacctttaccaattattggctgggttgggttaaacagcgtccgggtcatggtctggaatggattggtgatatttatccgggtggtggctataccaattataatgaaaaatttaaaggcaaagcaaccctgaccgcagataccagcagcagcaccgcatatatgcagctgtctagcctgaccagcgaagatagcgcagtttatttctgtgcacgcacaccgctgtatggtagcagctatggtggttttgcatattggggtcagggcaccctggttaccgttagcgcaggtggtggtggatccgatattcagatgacccagaccccgtctagcctgagcgcaagcctgggtgatcgtgttaccattagctgtagcgcaagccagggtattagcaattatctgaattggtatcagcagaaaccggatggcaccgttaaactgctgatttattataccagcagcctgcatagcggtgttccgagccgttttagcggtagcggttctggcaccgattatagcctgaccatttctaatctggaaccggaagatattgccacctattattgccagcagtatagcaaactgccgtggacctttggtggtggcaccaaactggagattaaataactcgag

SEQ ID NO: 30 - protein, 234 aa, Artificial sequence
  Synthetic Construct
QVQLQQSGAELVRPGTSVKISCKASGYTFTNYWLGWVKQRPGHGLEWIGDIYPGGGYTNYNEKFKGKATLTADTSSSTAYMQLSSLTSEDSAVYFCARTPLYGSSYGGFAYWGQGTLVTVSAGGGGSDIQMTQTPSSLSASLGDRVTISCSASQGISNYLNWYQQKPDGTVKLLIYYTSSLHSGVPSRFSGSGSGTDYSLTISNLEPEDIATYYCQQYSKLPWTFGGGTKLEIK